(19) United States
(12) Patent Application Publication
(10) Pub. No.: US 2010/0129403 A1
(43) Pub. Date: May 27, 2010

(54) RECOMBINANT VIRAL VACCINE

(75) Inventors: Jean-Yves Bonnefoy, Strasbourg (FR); Stephane Paul, Strasbourg (FR)

Correspondence Address: BUCHANAN, INGERSOLL & ROONEY PC, POST OFFICE BOX 1404, ALEXANDRIA, VA, 22313-1404, US

(73) Assignee: Transgene S.A., Strasbourg (FR)

(21) Appl. No.: 12/306,227

(22) PCT Filed: Jun. 15, 2007
(86) PCT No.: PCT/EP07/05303

§ 371 Date: Nov. 2, 2009

Related U.S. Application Data
(60) Provisional application No. 60/852,964, filed on Oct. 20, 2006.

(30) Foreign Application Priority Data
Jun. 20, 2006 (EP) 06360028.2

(51) Int. Cl. A61K 39/275; A61P 31/12; A61P 37/04

(52) U.S. Cl. 424/232.1

(57) ABSTRACT

The present invention concerns new recombinant viral vaccines. In particular the present invention provides combination products that comprise recombinant viral vectors and specific compounds able to improve the immune response raised in vivo by said recombinant viral vectors.

The present invention provides new recombinant viral vaccines. In particular the present invention provides combination products that comprise recombinant viral vectors and specific compounds able to improve the immune response raised in vivo by said recombinant viral vectors.

Traditional vaccination techniques involving the introduction into an animal system of an antigen which can induce an immune response, and thereby protect said animal against infection, have been known for many years. These techniques have included the development of both live and inactivated vaccines. Live vaccines are typically attenuated non-pathogenic versions of an infectious agent that are capable of priming an immune response directed against a pathogenic version of the infectious agent. In recent years there have been advances in the development of recombinant vaccines, especially recombinant live vaccines, in which foreign antigens of interest are encoded and expressed from a vector. Amongst them, vectors based on recombinant viruses have shown great promise and play an important role in the development of new vaccines. Many viruses have been investigated for their ability to express proteins from foreign pathogens or tumoral tissue, and to induce specific immunological responses against these antigens in vivo. Generally, these gene-based vaccines can stimulate potent humoral and cellular immune responses and viral vectors might be an effective strategy for both the delivery of antigen-encoding genes and the facilitation and enhancement of antigen presentation. In order to be utilized as a vaccine carrier, the ideal viral vector should be safe and enable efficient presentation of required pathogen-specific antigens to the immune system. It should also exhibit low intrinsic immunogenicity to allow for its re-administration in order to boost relevant specific immune responses. Furthermore, the vector system must meet criteria that enable its production on a large-scale basis. Several viral vaccine vectors have thus emerged to date, all of them having relative advantages and limits depending on the proposed application, and thus far none of them have proven to be ideal vaccine carriers.

Recombinant poxvirus vectors are examples of viral vaccine vectors. They have been used as inducers of both humoral and cellular immune responses, inducing both CD4+ and CD8+ T cells, and therefore represent a delivery system of choice especially in cancer or antiviral immunotherapy (see Arlen et al., 2005, Semin Oncol., 32, 549-555 or Essajee and Kaufman, 2004, Expert Opin Biol Ther., 4, 575-588). Despite the advantages associated with poxvirus vaccination relative to other vaccination therapies (see for example Souza et al, 2005, Braz J Med Biol Res, 38, 509-522), there is nonetheless a desire to develop adjuvant compounds adapted to this viral vector which will serve to increase the immune response induced by said vaccine.

There has been a major effort in recent years, with significant success, to discover new drug compounds that act by stimulating certain key aspects of the immune system. These compounds, referred as immune response modifiers (IRMs) or adjuvants, appear to act through basic immune system mechanisms via Toll-like receptors (TLRs) to induce various important cytokines biosynthesis (e.g., interferons, interleukins, tumor necrosis factor, etc.). Such compounds have been shown to stimulate a rapid release of certain monocyte/macrophage-derived cytokines and are also capable of stimulating B cells to secrete antibodies which play an important role in the antiviral and antitumor activities of IRM compounds. One of the predominant immunostimulating responses induced by IRMs is the induction of interferon IFN-alpha production, which is believed to be very important in the acute antiviral and antitumor activities seen. Moreover, up regulation of other cytokines such as, for example, tumor necrosis factor (TNF), IL-1 and IL-6 also have potentially beneficial activities and are believed to contribute to the antiviral and antitumor properties of these compounds. Immune response modifiers (IRMs) have been disclosed as useful for treating a wide variety of diseases and conditions, including viral diseases (e.g., human papilloma virus, hepatitis, herpes), neoplasias (e.g., basal cell carcinoma, squamous cell carcinoma, actinic keratosis, melanoma), and TH2-mediated diseases (e.g., asthma, allergic rhinitis, atopic dermatitis).

Examples of such immune response modifiers (IRMs), include the CpG oligonucleotides (see U.S. Pat. No. 6,194,388; US2006094683; WO 2004039829 for example), lipopolysaccharides, polyinosic:polycytidylic acid complexes (Kadowaki, et al., 2001, J. Immunol. 166, 2291-2295), and polypeptides and proteins known to induce cytokine production from dendritic cells and/or monocyte/macrophages. Other examples of such immune response modifiers (IRMs) are small organic molecule such as imidazoquinolinamines, imidazopyridine amines, 6,7-fused cycloalkylimidazopyridine amines, imidazonaphthyridine amines, oxazoloquinoline amines, thiazoloquinoline amines and 1,2-bridged imidazoquinoline amines (see for example U.S. Pat. No. 4,689,338; U.S. Pat. No. 5,389,640; U.S. Pat. No. 6,110,929; and U.S. Pat. No. 6,331,539).

In particular, the imidazoquinolinamines have demonstrated strong potency as inducers of interferon-alpha (IFN), tumor necrosis factor-alpha (TNF), interleukin IL-1 beta, IL-6, IL-1 alpha, IL-1 receptor antagonist, IL-10, granulocyte-macrophage colony-stimulating factor (GM-CSF), granulocyte CSF (G-CSF), and macrophage inflammatory protein-1 alpha in vitro and in vivo (Gibson et al., 1995, J Interferon Cytokine Res., 15, 537-545; Tomai et al., 1995, Antiviral Res., 28, 253-264; Testerman et al., 1995, J Leukoc Biol., 58, 365-372), and have been shown to cause diverse biological functions, involving antiviral, antiproliferative and antitumour activities (for review, see Syed, 2001, Expert Opin Pharmacother., 2, 877-882 or Li et al, 2005, J Drugs Dermatol., 4, 708-717). More particularly, inventors of patent application WO 93/20847 have shown that imidazoquinolinamines were able to enhance the immune response towards certain antigens such as live viral and bacterial immunogens, tumor-derived, protozoal, organism-derived, fungal and bacterial immunogens, toxoids, toxins, polysaccharides, proteins, glycoproteins, peptides and the like when these antigens were co-administered with this category of compounds. The antiviral activity of imidazoquinolinamine compounds has been further demonstrated against a variety of viruses, especially poxviruses (Bikowski, 2004, Cutis., 73, 202-206; US 20050048072), and their clinical efficacy has been demonstrated against genital warts (Scheinfeld and Lehman, 2006, Dermatol Online J., 12, 5), herpes genitalis (Miller et al, 2002, Int Immunopharmacol., 2, 443-451) and molluscum contagiosum (Stulberg and Galbraith Hutchinson, 2003, Am. Fam. Physician, 67, 1233-1240).

Following the observation in the early 1990's that plasmid DNA vectors could directly transfect animal cells in vivo, significant research efforts have been undertaken to develop vaccination techniques based upon the use of DNA plasmids to induce immune response, by direct introduction into animals of DNA which encodes for antigenic peptides. Such techniques which are widely referred as DNA vaccination have now been used to elicit protective immune responses in large number of disease models. More recently, imidazoquinolinamines have been proposed as adjuvants in DNA vaccination (WO 02/24225), especially in cancer immunotherapy (WO 2006/042254; Smorlesi et al, 2005, Gene Therapy, 12, 1324-1332). For a review on DNA vaccines, see Reyes-Sandoval and Ertl, 2001 (Current Molecular Medicine, 1, 217-243).

The present invention relates to an improvement to recombinant viral vaccines expressing in vivo at least one heterologous nucleotide sequence, especially a nucleotide sequence encoding an antigen. It relates in particular to a recombinant viral vaccine containing at least one recombinant viral vector expressing at least one antigen and at least one adjuvant which is capable of remarkably increasing the immunity conferred against said antigen relative to the same recombinant viral vaccine with no adjuvant and which is perfectly suitable for this type of vaccine. It further relates to vaccination methods relating thereto.

The Applicant has now surprisingly found that certain imidazoquinolinamine compounds while presenting strong antiviral potency were capable to improve the immune response raised by recombinant viral vaccines towards the antigen encoded by a recombinant viral vector, and more specifically for vaccine based on recombinant poxvirus vector, and this in unexpected proportions.

The subject of the present invention is therefore a recombinant viral vaccine containing (i) at least one recombinant viral vector expressing in vivo at least one heterologous nucleotide sequence, especially an heterologous nucleotide sequence encoding an antigen, and (ii) at least one 1H-imidazo[4,5-c]quinolin-4-amine derivative.

According to one embodiment of the present invention, said 1H-imidazo[4,5-c]quinolin-4-amine derivative enhances the immune responses in a patient to the said antigen where the said recombinant viral vaccine is administered to said patient.

As used herein throughout the entire application, the terms “a” and “an” are used in the sense that they mean “at least one”, “at least a first”, “one or more” or “a plurality” of the referenced compounds or steps, unless the context dictates otherwise. For example, the term “a cell” includes a plurality of cells including a mixture thereof. More specifically, “at least one” and “one or more” means a number which is one or greater than one, with a special preference for one, two or three.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

As used herein, the term “comprising”, “containing” when used to define products, compositions and methods, is intended to mean that the products, compositions and methods include the referenced compounds or steps, but not excluding others.

The term “patient” refers to a vertebrate, particularly a member of the mammalian species and includes, but is not limited to, domestic animals, sport animals, primates including humans. The term “patient” is in no way limited to a special disease status, it encompasses both patients who have already developed a disease of interest and patients who are not sick.

As used herein, the term “treatment” or “treating” encompasses prophylaxis and/or therapy. Accordingly the recombinant viral vaccines of the present invention are not limited to therapeutic applications and can be used in prophylaxis ones.

According to a more preferred embodiment, the recombinant viral vector according to the present invention is a poxviral vector (see for example Cox et al. in “Viruses in Human Gene Therapy” Ed J. M. Hos, Carolina Academic Press). According to another preferred embodiment it is selected in the group consisting of vaccinia virus, suitable vaccinia viruses include without limitation the Copenhagen strain (Goebel et al., 1990, Virol. 179, 247-266 and 517-563; Johnson et al., 1993, Virol. 196, 381-401), the Wyeth strain and the highly attenuated attenuated virus derived thereof including MVA (for review see Mayr, A., et al., 1975, Infection 3, 6-14) and derivates thereof (such as MVA vaccinia strain 575 (ECACC V00120707—U.S. Pat. No. 6,913,752), NYVAC (see WO 92/15672—Tartaglia et al., 1992, Virology, 188, 217-232). It may also be obtained from any other member of the poxviridae, in particular fowlpox (e.g. TROVAC, see Paoletti et al, 1995, Dev Biol Stand., 84, 159-163); canarypox (e.g. ALVAC, WO 95/27780, Paoletti et al, 1995, Dev Biol Stand., 84, 159-163); pigeonpox; swinepox and the like. By way of example, persons skilled in the art may refer to WO 92 15672 (incorporated by reference) which describes the production of expression vectors based on poxviruses capable of expressing such heterologous nucleotide sequence, especially nucleotide sequence encoding antigen.

As used herein, the term “antigen” refers to any substance that is capable of being the target of an immune response. An antigen may be the target of, for example, a cell-mediated and/or humoral immune response raised by a patient. The term “antigen” encompasses for example viral antigens, tumour-specific or -related antigens, bacterial antigens, parasitic antigens, allergens and the like:

Viral antigens include for example antigens from hepatitis viruses A, B, C, D & E, HIV, herpes viruses, cytomegalovirus, varicella zoster, papilloma viruses, Epstein Barr virus, influenza viruses, para-influenza viruses, adenoviruses, coxsakie viruses, picorna viruses, rotaviruses, respiratory syncytial viruses, pox viruses, rhinoviruses, rubella virus, papovirus, mumps virus, measles virus; some non-limiting examples of known viral antigens include the following: antigens derived from HIV-1 such as tat, nef, gp120 or gp160, gp40, p24, gag, env, vif, vpr, vpu, rev or part and/or combinations thereof; antigens derived from human herpes viruses such as gH, gL gM gB gC gK gE or gD or or part and/or combinations thereof or Immediate Early protein such asICP27, ICP47, ICP4, ICP36 from HSV1 or HSV2; antigens derived from cytomegalovirus, especially human cytomegalovirus such as gB or derivatives thereof; antigens derived from Epstein Barr virus such as gp350 or derivatives thereof; antigens derived from Varicella Zoster Virus such asgpl, 11, 111 and IE63; antigens derived from a hepatitis virus such as hepatitis B, hepatitis C or hepatitis E virus antigen (e.g. env protein E1 or E2, core protein, NS2, NS3, NS4a, NS4b, NS5a, NS5b, p7, or part and/or combinations thereof of HCV); antigens derived from human papilloma viruses (for example HPV6,11,16,18, e.g. L1, L2, E1 , E2, E3, E4, E5, E6, E7, or part and/or combinations thereof); antigens derived from other viral pathogens, such as Respiratory Syncytial virus (e.g F and G proteins or derivatives thereof), parainfluenza virus, measles virus, mumps virus, flaviviruses (e. g. Yellow Fever Virus, Dengue Virus, Tick-borne encephalitis virus, Japanese Encephalitis Virus) or Influenza virus cells (e.g. HA, NP, NA, or M proteins, or part and/or combinations thereof);

tumor-specific or -related antigens includes for example antigens from breast cancer, colon cancer, rectal cancer, cancer of the head and neck, renal cancer, malignant melanoma, laryngeal cancer, ovarian cancer, cervical cancer, prostate cancer. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Some non-limiting examples of tumor-specific or -related antigens include MART-1/Melan-A, gp100, Dipeptidyl peptidase IV (DPPIV), adenosine deaminase-binding protein (ADAbp), cyclophilin b, Colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family (e.g. MUC-1), HER2/neu, p21ras, RCAS1, alpha-fetoprotein, E-cadherin, alpha-catenin, beta-catenin and gamma-catenin, p120ctn, gp100.sup.Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2;

bacterial antigens includes for example antigens from Mycobacteria causing TB and leprosy, pneumocci, aerobic gram negative bacilli, mycoplasma, staphyloccocal infections, streptococcal infections, salmonellae, chlamydiae, neisseriae;

other antigens includes for example antigens from malaria, leishmaniasis, trypanosomiasis, toxoplasmosis, schistosomiasis, filariasis;

allergens refer to a substance that can induce an allergic or asthmatic response in a susceptible subject. The list of allergens is enormous and can include pollens, insect venoms, animal dander dust, fungal spores and drugs (e.g. penicillin). Examples of natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

In a particularly preferred embodiment the heterologous nucleotide sequence of the present invention, encodes one or more of all or part of the following antigens HBV-PreS1 PreS2 and Surface env proteins, core and polHIV-gp120 gp40,gp160, p24, gag, pol, env, vif, vpr, vpu, tat, rev, nef; HPV-E1, E2, E3, E4, E5, E6, E7, E8, L1, L2 (see for example WO 90/10459, WO 98/04705, WO 99/03885); HCV env protein E1 or E2, core protein, NS2, NS3, NS4a, NS4b, NS5a, NS5b, p7; Muc-1 (see for example U.S. Pat. No. 5,861,381; U.S. Pat. No. 6,054,438; WO98/04727; WO98/37095).

The nucleic acid encoding the antigen is operatively linked to a gene expression sequence which directs the expression of the antigen nucleic acid within a eukaryotic cell. The gene expression sequence is any regulatory nucleotide sequence, such as a promoter sequence or promoter-enhancer combination, which facilitates the efficient transcription and translation of the antigen nucleic acid to which it is operatively linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, b-actin promoter and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art. In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined antigen nucleic acid. The gene expression sequences optionally include enhancer sequences or upstream activator sequences as desired. Preferred promoters for use in a poxviral vector (see below) include without limitation vaccinia promoters 7.5K, H5R, TK, p28, p11 and K1L, chimeric promoters between early and late poxviral promoters as well as synthetic promoters such as those described in Chakrabarti et al. (1997, Biotechniques 23, 1094-1097), Hammond et al. (1997, J. Virological Methods 66, 135-138) and Kumar and Boyle (1990, Virology 179, 151-158).

According to another special embodiment, said heterologous nucleotide sequence of the present invention, encodes all or part of HPV antigen(s) selected in the group consisting of E6 early coding region of HPV, E7 early coding region of HPV and derivates or combination thereof.

The HPV antigen encoded by the recombinant viral vector according to the invention is selected in the group consisting of an HPV E6 polypeptide, an HPV E7 polypeptide or both an HPV E6 polypeptide and an HPV E7 polypeptide. The present invention encompasses the use of any HPV E6 polypeptide which binding to p53 is altered or at least significantly reduced and/or the use of any HPV E7 polypeptide which binding to Rb is altered or at least significantly reduced (Munger et al., 1989, EMBO J. 8, 4099-4105; Crook et al., 1991, Cell 67, 547-556; Heck et al., 1992, Proc. Natl. Acad. Sci. USA 89, 4442-4446; Phelps et al., 1992, J. Virol. 66, 2148-2427). A non-oncogenic HPV-16 E6 variant which is suitable for the purpose of the present invention is deleted of one or more amino acid residues located from approximately position 118 to approximately position 122 (+1 representing the first methionine residue of the native HPV-16 E6 polypeptide), with a special preference for the complete deletion of residues 118 to 122 (CPEEK). A non-oncogenic HPV-16 E7 variant which is suitable for the purpose of the present invention is deleted of one or more amino acid residues located from approximately position 21 to approximately position 26 (+1 representing the first amino acid of the native HPV-16 E7 polypeptide, with a special preference for the complete deletion of residues 21 to 26 (DLYCYE). According to a preferred embodiment, the one or more HPV-16 early polypeptide(s) in use in the invention is/are further modified so as to improve MHC class I and/or MHC class II presentation, and/or to stimulate anti-HPV immunity. HPV E6 and E7 polypeptides are nuclear proteins and it has been previously shown that membrane presentation permits to improve their therapeutic efficacy (see for example WO99/03885). Thus, it may be advisable to modify at least one of the HPV early polypeptide(s) so as to be anchored to the cell membrane. Membrane anchorage can be easily achieved by incorporating in the HPV early polypeptide a membrane-anchoring sequence and if the native polypeptide lacks it a secretory sequence (i.e. a signal peptide). Membrane-anchoring and secretory sequences are known in the art. Briefly, secretory sequences are present at the N-terminus of the membrane presented or secreted polypeptides and initiate their passage into the endoplasmic reticulum (ER). They usually comprise 15 to 35 essentially hydrophobic amino acids which are then removed by a specific ER-located endopeptidase to give the mature polypeptide. Membrane-anchoring sequences are usually highly hydrophobic in nature and serves to anchor the polypeptides in the cell membrane (see for example Branden and Tooze, 1991, in Introduction to Protein Structure p. 202-214, NY Garland).

The choice of the membrane-anchoring and secretory sequences which can be used in the context of the present invention is vast. They may be obtained from any membrane-anchored and/or secreted polypeptide comprising it (e.g. cellular or viral polypeptides) such as the rabies glycoprotein, of the HIV virus envelope glycoprotein or of the measles virus F protein or may be synthetic. The membrane anchoring and/or secretory sequences inserted in each of the early HPV-16 polypeptides used according to the invention may have a common or different origin. The preferred site of insertion of the secretory sequence is the N-terminus downstream of the codon for initiation of translation and that of the membrane-anchoring sequence is the C-terminus, for example immediately upstream of the stop codon.

The HPV E6 polypeptide in use in the present invention is preferably modified by insertion of the secretory and membrane-anchoring signals of the measles F protein. Optionally or in combination, the HPV E7 polypeptide in use in the present invention is preferably modified by insertion of the secretory and membrane-anchoring signals of the rabies glycoprotein.

The therapeutic efficacy of the recombinant viral vector can also be improved by using one or more nucleic acid encoding immunopotentiator polypeptide(s). For example, it may be advantageous to link the HPV early polypeptide(s) to a polypeptide such as calreticulin (Cheng et al., 2001, J. Clin. Invest. 108, 669-678), Mycobacterium tuberculosis heat shock protein 70 (HSP70) (Chen et al., 2000, Cancer Res. 60, 1035-1042), ubiquitin (Rodriguez et al., 1997, J. Virol. 71, 8497-8503) or the translocation domain of a bacterial toxin such as Pseudomonas aeruginosa exotoxin A (ETA(dIII)) (Hung et al., 2001 Cancer Res. 61, 3698-3703).

According to another and preferred embodiment, the recombinant viral vector according to the invention comprises a nucleic acid encoding one or more early polypeptide(s) as above defined, and more particularly HPV-16 and/or HPV-18 early E6 and/or E7 polypeptides.

According to another special embodiment, said heterologous nucleotide sequence of the present invention, encodes all or part of MUC 1 antigen or derivates thereof.

If needed, the nucleic acid molecule in use in the invention may be optimized for providing high level expression of the antigen (e.g. HPV early polypeptide(s)) in a particular host cell or organism, e.g. a human host cell or organism. Typically, codon optimisation is performed by replacing one or more “native” (e.g. HPV) codon corresponding to a codon infrequently used in the mammalian host cell by one or more codon encoding the same amino acid which is more frequently used. This can be achieved by conventional mutagenesis or by chemical synthetic techniques (e.g. resulting in a synthetic nucleic acid). It is not necessary to replace all native codons corresponding to infrequently used codons since increased expression can be achieved even with partial replacement. Moreover, some deviations from strict adherence to optimised codon usage may be made to accommodate the introduction of restriction site(s).

As mentioned above, poxviral vector is preferred, and more specifically highly attenuated vaccinia virus strains. Determination of the complete sequence of the MVA genome and comparison with the Copenhagen vaccinia virus genome has allowed the precise identification of the seven deletions (I to VII) which occurred in the MVA genome (Antoine et al., 1998, Virology 244, 365-396), any of which can be used to insert the antigen (e.g. HPV early polypeptide or MUC1)—encoding nucleic acid.

The basic technique for inserting the nucleic acid and associated regulatory elements required for expression in a poxviral genome is described in numerous documents accessible to the man skilled in the art (Paul et al., 2002, Cancer gene Ther. 9, 470-477; Piccini et al., 1987, Methods of Enzymology 153, 545-563; U.S. Pat. No. 4,769,330; U.S. Pat. No. 4,772,848; U.S. Pat. No. 4,603,112; U.S. Pat. No. 5,100,587 and U.S. Pat. No. 5,179,993). Usually, one proceed through homologous recombination between overlapping sequences (i.e. desired insertion site) present both in the viral genome and a plasmid carrying the nucleic acid to insert.

The nucleic acid encoding the antigen of the Invention is preferably inserted in a nonessential locus of the poxviral genome, in order that the recombinant poxvirus remains viable and infectious. Nonessential regions are non-coding intergenic regions or any gene for which inactivation or deletion does not significantly impair viral growth, replication or infection. One may also envisage insertion in an essential viral locus provided that the defective function be supplied in trans during production of viral particles, for example by using an helper cell line carrying the complementing sequences corresponding to those deleted in the poxviral genome.

When using the Copenhagen vaccinia virus, the antigen-encoding nucleic acid is preferably inserted in the thymidine kinase gene (tk) (Hruby et al., 1983, Proc. Natl. Acad. Sci USA 80, 3411-3415; Weir et al., 1983, J. Virol. 46, 530-537). However, other insertion sites are also appropriate, e.g. in the hemagglutinin gene (Guo et al., 1989, J. Virol. 63, 4189-4198), in the K1L locus, in the u gene (Zhou et al., 1990, J. Gen. Virol. 71, 2185-2190) or at the left end of the vaccinia virus genome where a variety of spontaneous or engineered deletions have been reported in the literature (Altenburger et al., 1989, Archives Virol. 105, 15-27; Moss et al. 1981, J. Virol. 40, 387-395; Panicali et al., 1981, J. Virol. 37, 1000-1010; Perkus et al, 1989, J. Virol. 63, 3829-3836; Perkus et al, 1990, Virol. 179, 276-286; Perkus et al, 1991, Virol. 180, 406-410).

When using MVA, the antigen-encoding nucleic acid can be inserted in anyone of the identified deletions I to VII as well as in the D4R locus, but insertion in deletion II or III is preferred (Meyer et al., 1991, J. Gen. Virol. 72, 1031-1038; Sutter et al., 1994, Vaccine 12, 1032-1040).

When using fowlpox virus, although insertion within the thymidine kinase gene may be considered, the antigen-encoding nucleic acid is preferably introduced in the intergenic region situated between ORFs 7 and 9 (see for example EP 314 569 and U.S. Pat. No. 5,180,675).

Preferably, the antigen-encoding nucleic acid in use in the invention is in a form suitable for its expression in a host cell or organism, which means that the nucleic acid sequence encoding the antigen (e.g. E6 polypeptide and/or the nucleic acid sequence encoding the E7 polypeptide) are placed under the control of one or more regulatory sequences necessary for expression in the host cell or organism. As used herein, the term “regulatory sequence” refers to any sequence that allows, contributes or modulates the expression of a nucleic acid in a given host cell, including replication, duplication, transcription, splicing, translation, stability and/or transport of the nucleic acid or one of its derivative (i.e. mRNA) into the host cell. It will be appreciated by those skilled in the art that the choice of the regulatory sequences can depend on factors such as the host cell, the vector and the level of expression desired.

The promoter is of special importance and the present invention encompasses the use of constitutive promoters which direct expression of the nucleic acid in many types of host cell and those which direct expression only in certain host cells or in response to specific events or exogenous factors (e.g. by temperature, nutrient additive, hormone or other ligand). Suitable promoters are widely described in literature and one may cite more specifically viral promoters such as RSV, SV40, CMV and MLP promoters. Preferred promoters for use in a poxviral vector include without limitation vaccinia promoters 7.5K, H5R, TK, p28, p11 and K1L, chimeric promoters between early and late poxviral promoters as well as synthetic promoters such as those described in Chakrabarti et al. (1997, Biotechniques 23, 1094-1097), Hammond et al. (1997, J. Virological Methods 66, 135-138) and Kumar and Boyle (1990, Virology 179, 151-158).

Those skilled in the art will appreciate that the regulatory elements controlling the expression of the nucleic acid molecule of the invention may further comprise additional elements for proper initiation, regulation and/or termination of transcription (e.g. polyA transcription termination sequences), mRNA transport (e.g. nuclear localization signal sequences), processing (e.g. splicing signals), and stability (e.g. introns and non-coding 5′ and 3′ sequences), translation (e.g. peptide signal, propeptide, tripartite leader sequences, ribosome binding sites, Shine-Dalgamo sequences, etc.) into the host cell or organism.

Alternatively, the recombinant viral vector in use in the present invention can further comprise at least one nucleic acid encoding at least one cytokine. Suitable cytokines include without limitation IL-2, IL-7, IL-15, IL-18, IL-21 and IFNg, with a special preference for IL-2. When the recombinant viral vaccine of the invention comprises a cytokine-expressing nucleic acid, said nucleic acid may be carried by the recombinant viral vector encoding the one or more HPV early polypeptide(s) or by an independent recombinant vector which can be of the same or a different origin.

A preferred embodiment of the invention is directed to the use of a recombinant viral vaccine comprising a MVA vector encoding the HPV E6 polypeptide placed under the 7.5K promoter, the HPV E7 polypeptide placed under the 7.5K promoter and the human IL-2 gene placed under the control of the H5R promoter. Preferably, nucleic acids encoding the HPV E6 polypeptide, the HPV E7 polypeptide and the human IL-2 are inserted in deletion III of the MVA genome.

Another preferred embodiment of the invention is directed to the use of a recombinant viral vaccine comprising a MVA vector encoding the MUC 1 polypeptide placed under the 7.5K promoter, and the human IL-2 gene placed under the control of the H5R promoter.

Infectious viral particles comprising the above-described recombinant viral vector can be produced by routine process. An exemplary process comprises the steps of:

a. introducing the viral vector into a suitable cell line,

b. culturing said cell line under suitable conditions so as to allow the production of said infectious viral particle,

c. recovering the produced infectious viral particle from the culture of said cell line, and

d. optionally purifying said recovered infectious viral particle.

Cells appropriate for propagating poxvirus vectors are avian cells, and most preferably primary chicken embryo fibroblasts (CEF) prepared from chicken embryos obtained from fertilized eggs.

The infectious viral particles may be recovered from the culture supernatant or from the cells after lysis (e.g. by chemical means, freezing/thawing, osmotic shock, mechanic shock, sonication and the like). The viral particles can be isolated by consecutive rounds of plaque purification and then purified using the techniques of the art (chromatographic methods, ultracentrifugation on cesium chloride or sucrose gradient).

According to another embodiment, the 1H-imidazo[4,5-c]quinolin-4-amine-derivative of the present invention is a compound defined by one of the following general formulae I-V:

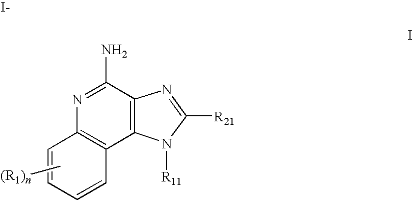

or analogues, solvates or salts thereof,

wherein

R₁₁ is selected from the group consisting of straight or branched alkyl, hydroxyalkyl, acyloxyalkyl, benzyl, (phenyl)ethyl and phenyl, said benzyl, (phenyl)ethyl or phenyl substituent being optionally substituted on the benzene ring by one or two moieties selected, independently from one another, from the group consisting of C₁₋₄ alkyl moiety, C₁₋₄ alkoxy moiety and halogen, with the proviso that if said benzene ring is substituted by two of said moieties, then said moieties together contain no more than 6 carbon atoms;

R₂₁ is selected from the group consisting of hydrogen, C₁₋₈ alkyl moiety, benzyl, (phenyl)ethyl and phenyl, the benzyl, (phenyl)ethyl or phenyl substituent being optionally substituted on the benzene ring by one or two moieties selected, independently from one another, from the group consisting of C₁₋₄ alkyl moiety, C₁₋₄ alkoxy moiety and halogen, with the proviso that when the benzene ring is substituted by two of said moieties, then the moieties together contain no more than 6 carbon atoms;

and each R₁ is selected, independently from one another, from the group consisting of hydrogen, C₁₋₄ alkoxy moiety, halogen and C₁₋₄ alkyl moiety, and n is an integer from 0 to 2, with the proviso that if n is 2, then said R₁ groups together contain no more than 6 carbon atoms;

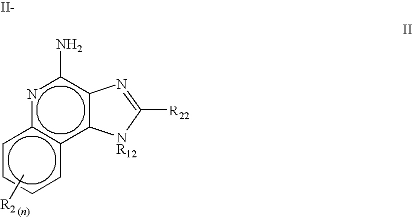

or analogues, solvates or salts thereof,

wherein

R₁₂ is selected from the group consisting of straight or branched C₂₋₁₀ alkenyl and substituted straight or branched C₂₋₁₀ alkenyl, wherein the substituent is selected from the group consisting of straight or branched C₁₋₄ alkyl moiety and C₃₋₆ cycloalkyl moiety; and C₃₋₆ cycloalkyl moiety substituted by straight or branched C₁₋₄ alkyl moiety; and

R₂₂ is selected from the group consisting of hydrogen, straight or branched C₁₋₈ alkyl moiety, benzyl, (phenyl)ethyl and phenyl, the benzyl, (phenyl)ethyl or phenyl substituent being optionally substituted on the benzene ring by one or two moieties selected, independently from one another, from the group consisting of straight or branched C₁₋₄ alkyl moiety, straight or branched C₁₋₄ alkoxy moiety, and halogen, with the proviso that when the benzene ring is substituted by two such moieties, then the moieties together contain no more than 6 carbon atoms;

and each R₂ is selected, independently from one another, from the group consisting of straight or branched C₁₋₄ alkoxy moiety, halogen, and straight or branched C₁₋₄ alkyl moiety, and n is an integer from zero to 2, with the proviso that if n is 2, then said R₂ groups together contain no more than 6 carbon atoms;

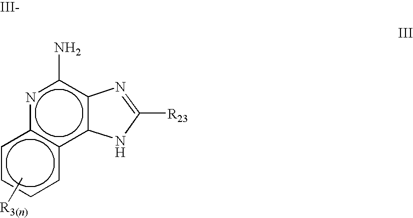

or analogues, solvates or salts thereof,

wherein

R₂₃ is selected from the group consisting of hydrogen, straight or branched C₁₋₈ alkyl moiety, benzyl, (phenyl)ethyl and phenyl, the benzyl, (phenyl)ethyl or phenyl substituent being optionally substituted on the benzene ring by one or two moieties selected, independently from one another, from the group consisting of straight or branched C₁₋₄ alkyl moiety, straight or branched C₁₋₄ alkoxy moiety, and halogen, with the proviso that when the benzene ring is substituted by two such moieties, then the moieties together contain no more than 6 carbon atoms;

and each R₃ is selected, independently from one another, from the group consisting of straight or branched C₁₋₄ alkoxy moiety, halogen, and straight or branched C₁₋₄ alkyl moiety, and n is an integer from zero to 2, with the proviso that if n is 2, then said R₃ groups together contain no more than 6 carbon atoms;

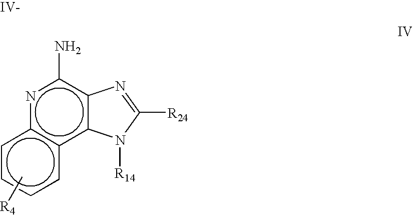

or analogues, solvates or salts thereof,

wherein

R₁₄ is —CHR₃₄R₄₄ wherein R₄₄ is hydrogen or a carbon-carbon bond, with the proviso that when R₄₄ is hydrogen R₃₄ is C₁₋₄ alkoxy moiety, C₁₋₄ hydroxyalkoxy moiety, C₂₋₁₀ 1-alkynyl moiety, tetrahydropyranyl, alkoxyalkyl wherein the alkoxy moiety contains one to four carbon atoms and the alkyl moiety contains one to four carbon atoms, 2-, 3-, or 4-pyridyl, and with the further proviso that when R₄₄ is a carbon-carbon bond R₄₄ and R₃₄ together form a tetrahydrofuranyl group optionally substituted with one or more substituents selected, independently from one another, from the group consisting of hydroxy and C₁₋₄hydroxyalkyl moities;

R₂₄ is selected from the group consisting of hydrogen, C₁₋₄ alkyl, phenyl, wherein the phenyl is optionally substituted by one or two moieties selected, independently from one another, from the group consisting of straight or branched C₁₋₄ alkyl moiety, straight or branched C₁₋₄ alkoxy moiety, and halogen;

and R₄ is selected from the group consisting of hydrogen, straight or branched C₁₋₄ alkoxy moiety, halogen, and straight or branched C₁₋₄ alkyl moiety;

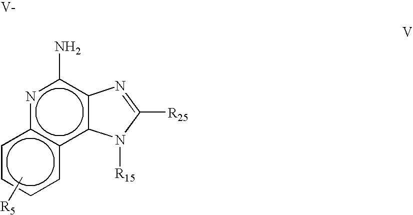

or analogues, solvates or salts thereof,

wherein

R₁₅ is selected from the group consisting of hydrogen; straight or branched C₁₋₁₀ alkyl moiety and substituted straight or branched C₁₋₁₀ alkyl moiety, wherein the substituent is selected from the group consisting of C₃₋₆ cycloalkyl and C₃₋₆ cycloalkyl substituted by straight or branched C₁₋₄ alkyl moiety; straight or branched C₂₋₁₀ alkenyl and substituted straight or branched C₂₋₁₀ alkenyl moiety, wherein the substituent is selected from the group consisting of C₃₋₆ cycloalkyl and C₃₋₆ cycloalkyl substituted by straight or branched C₁₋₄ alkyl moiety; C₁₋₆ hydroxyalkyl; alkoxyalkyl wherein the alkoxy moiety contains one to about four carbon atoms and the alkyl moiety contains one to about six carbon atoms; acyloxyalkyl wherein the acyloxy moiety is alkanoyloxy of two to about four carbon atoms or benzoyloxy, and the alkyl moiety contains one to about six carbon atoms; benzyl; (phenyl)ethyl; and phenyl; said benzyl, (phenyl)ethyl or phenyl substituent being optionally substituted on the benzene ring by one or two moieties selected, independently from one another, from the group consisting of C₁₋₄ alkyl moiety, C₁₋₄ alkoxy moiety, and halogen, with the proviso that when said benzene ring is substituted by two of said moieties, then the moieties together contain no more than six carbon atoms;

R₂₅ is

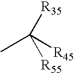

wherein

R₃₅ is selected from the group consisting of C₁₋₄ alkoxy moiety, alkoxyalkyl wherein the alkoxy moiety contains one to about four carbon atoms and the alkyl moiety contains one to about four carbon atoms; C₁₋₄ haloalkyl moiety; alkylamido wherein the alkyl group contains one to about four carbon atoms; amino; amino substituted with C₁₋₄ alkyl or C₁₋₄ hydroxyalkyl; azido; C₁₋₄ alkylthio;

R₅₅ and R₄₅ are selected, independently from one another, from the group consisting of hydrogen, C₁₋₄ alkyl moiety, phenyl, wherein said phenyl is optionally substituted by one or two moieties selected, independently from one another, from the group consisting of straight or branched C₁₋₄ alkyl moiety, straight or branched C₁₋₄ alkoxy moiety, and halogen; and

R₅ is selected from the group consisting of hydrogen, straight or branched C₁₋₄ alkoxy moiety, halogen, and straight or branched C₁₋₄ alkyl containing moiety.

According to special embodiment, the C₁₋₄ alkyl moiety is for example methyl, ethyl, propyl, 2-methylpropyl and butyl. According to preferred embodiment, the C₁₋₄ alkyl moiety is selected in the group consisting in methyl, ethyl and 2methyl-propyl.

According to special embodiment, the alkoxy moiety is selected in the group consisting in methoxy, ethoxy and ethoxymethyl.

According to preferred embodiment, n is zero or one.

According to preferred embodiment, R₁-R₅ groups are hydrogen.

According to preferred embodiment, R₁₁-R₁₅ groups are selected in the group consisting in 2-methylpropyl and 2-hydroxy-2-methylpropyl.

According to preferred embodiment, R₂₁-R₂₅ groups are selected in the group consisting in hydrogen, C₁₋₆ alkyl moiety, alkoxyalkyl wherein the alkoxy moiety contains one to about four carbon atoms and the alkyl moiety contains one to about four carbon atoms. Most preferred R₂₁-R₂₅ groups are selected in the group consisting in hydrogen, methyl, or ethoxymethyl.

According to one preferred embodiment, the 1H-imidazo[4,5-c]quinolin-4-amine-derivative of the present invention is a compound defined the following general formula VI:

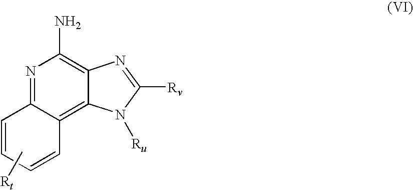

or analogues, solvates or salts thereof,

wherein

Rt is selected from the group consisting of hydrogen, straight or branched C₁₋₄ alkoxy moiety, halogen, and straight or branched C₁₋₄ alkyl;

Ru is 2-methylpropyl or 2-hydroxy-2-methylpropyl; and

Rv is hydrogen, C₁₋₆ alkyl, or alkoxyalkyl wherein the alkoxy moiety contains one to about four carbon atoms and the alkyl moiety contains one to about four carbon atoms.

According to preferred embodiment, in formula VI, Rt is hydrogen, Ru is 2-methylpropyl or 2hydroxy-2-methylpropyl, and Rv is hydrogen, methyl or ethoxymethyl.

According to another preferred embodiment, the 1H-imidazo[4,5-c]quinolin-4-amine-derivative of the present invention is a compound selected in the following group:

1-(2-methylpropyl)-1H-imidazo[4,5-c]quinolin-4-amine (a compound of formula VI wherein Rt is hydrogen, Ru is 2-methylpropyl and Rv is hydrogen);

1-(2-hydroxy-2-methylpropyl)-2-methyl-1H-imidazo[4,5-c]quinolin-4-amine (a compound of formula VI wherein Rt is hydrogen, Ru is 2-hydroxy-2-methylpropyl, and Rv is methyl;

1-(2-hydroxy-2-methylpropyl)-1H-imidazo[4,5-c]quinolin-4-amine (a compound of formula VI wherein Rt is hydrogen, Ru is 2-hydroxy-2-methylpropyl, and Rv is hydrogen);

1-(2-hydroxy-2-methylpropyl-2-ethoxymethyl-1-H-imidazo[4,5-c]quinolin-4-amine (a compound of formula VI wherein Rt is hydrogen, Ru is 2-hydroxy-2-methylpropyl and Rv is ethoxymethyl);

or analogues, solvates or salts thereof.

Persons skilled in the art can refer for example to U.S. Pat. No. 4,689,338, U.S. Pat. No. 4,929,624, EP 0385630 or WO 94/17043 (incorporated herein by reference) which describes the compounds recited above and methods for their preparation.

More specifically, the 1-(2-methylpropyl)-1H-imidazo[4,5-c]quinolin-4-amine (also known by the term imiquimod or Aldara) has been widely disclosed, reference may be made to Buck, 1998, Infect. Dis. Obstet. Gynecol., 6, 49-51; Dockrell and Kinghorn, 2001, J. Antimicrob. Chemother., 48, 751-755 or Garland, 2003, Curr. Opin. Infect. Dis., 16, 85-89; the 1-(2-hydroxy-2-methylpropyl)-2-methyl-1H-imidazo[4,5-c]quinolin-4-amine and the 1-(2-hydroxy-2-methylpropyl)-1H-imidazo[4,5-c]quinolin-4-amine have been disclosed in US 2004/0076633, and 1-(2-hydroxy-2-methylpropyl)-2-ethoxymethyl-1-H-imidazo[4,5-c]quinolin-4-amine (also known by the term resiquimod) in Dockrell and Kinghorn, 2001, J. Antimicrob. Chemother. 48, 751-755 or Jones, Curr. Opin. Investig. Drugs., 2003, 4, 214-218.

Unless otherwise indicated, reference to a 1H-imidazo[4,5-c]quinolin-4-amine-derivative can include the compound in any pharmaceutically acceptable form, including any isomer (e. g., diastereomer or enantiomer), salt, solvate, polymorph, and the like. In particular, if a compound is optically active, reference to the compound can include each of the compound's enantiomers as well as racemic mixtures of the enantiomers.

According to one preferred embodiment, the recombinant viral vaccine and more particularly the recombinant viral vector does not comprise an immunostimulatory motif or backbone that induces by itself an immune response especially a nucleotide sequence that possess immunostimulatory motif or backbone such as CpG, polyG, polyT, TG, methylated CpG, CpI and T rich motif or phosphorothioate backbones (see US 2003/0139364, U.S. Pat. No. 6,207,646 or WO 01/22972 the content of which is incorporated herein by reference.

According to one embodiment, the 1H-imidazo[4,5-c]quinolin-4-amine derivative concentration in the final recombinant viral vaccine will be from about 0.0001% to about 10% (unless otherwise indicated, all percentages provided herein are weight/weight with respect to the total formulation), from about 0.01% to about 2%, more particularly from about 0.06 to about 1%, preferably from about 0.1 to about 0.6%.

According to another embodiment, the appropriate dosage of recombinant viral vector can be adapted as a function of various parameters, in particular the mode of administration; the composition employed; the age, health, and weight of the host organism; the nature and extent of symptoms; kind of concurrent treatment; the frequency of treatment; and/or the need for prevention or therapy. Further refinement of the calculations necessary to determine the appropriate dosage for treatment is routinely made by a practitioner, in the light of the relevant circumstances. For general guidance, suitable dosage for a MVA-containing composition varies from about 10⁴ to 10¹⁰ pfu (plaque forming units), desirably from about 10⁵ and 10⁸ pfu whereas adenovirus-comprising composition varies from about 10⁵ to 10¹³ iu (infectious units), desirably from about 10⁷ and 10¹² iu. A composition based on vector plasmids may be administered in doses of between 10 μg and 20 mg, advantageously between 100 μg and 2 mg. Preferably the composition is administered at dose(s) comprising from 5×10⁵ pfu to 5×10⁷ pfu of MVA vaccinia vector.

The dosing regimen may depend at least in part on many factors known in the art including but not limited to the nature of the imidazo[4,5-c]quinolin-4-amine derivative and recombinant viral vector used, the nature of the carrier, the amount of the imidazo[4,5-c]quinolin-4-amine derivative and recombinant viral vector being administered, the state of the subject's immune system (e.g., suppressed, compromised, stimulated), and the method of administering the imidazo[4,5-c]quinolin-4-amine derivative and/or of recombinant viral vector compounds. Accordingly it is not practical to set forth generally the dosing regimen effective for increasing the efficacy of a recombinant viral vaccine for all possible applications. Those of ordinary skill in the art, however, can readily determine an appropriate dosing regimen with due consideration of such factors. In some embodiments of the invention, the imidazo[4,5-c]quinolin-4-amine derivative and/or recombinant viral vector compounds may be administered, for example, once to about once daily, although in some embodiments the imidazo[4,5-c]quinolin-4-amine derivative and/or recombinant viral vector compounds may be administered at a frequency outside this range. In certain embodiments, the imidazo[4,5-c]quinolin-4-amine derivative and/or recombinant viral vector compounds may be administered from about once per week to about once per day. In one particular embodiment, the imidazo[4,5-c]quinolin-4-amine derivative and/or recombinant viral vector compounds are administered once every weeks. Desirably, the imidazo[4,5-c]quinolin-4-amine derivative and recombinant viral vector is administered 1 to 10 times at weekly intervals. Preferably, the imidazo[4,5-c]quinolin-4-amine derivative and recombinant viral vector, or any composition containing it, is administered 3 times at weekly intervals by subcutaneous route.

In a further aspect, the invention provides a method of increasing an immune response to an antigen in a patient, said method comprising administration, either sequentially or simultaneously, of (i) a recombinant viral vector expressing in vivo at least one heterologous nucleotide sequence, especially an heterologous nucleotide sequence encoding an antigen and (ii) an imidazo[4,5-c]quinolin-4-amine derivative.

In another aspect, the invention provides a method of preventing occurrence of and/or of treating cancer in a patient, said method comprising administration, either sequentially or simultaneously, of (i) a recombinant viral vector expressing in vivo at least one heterologous nucleotide sequence, especially an heterologous nucleotide sequence encoding an antigen and (ii) an imidazo[4,5-c]quinolin-4-amine derivative.

In another aspect, the invention provides a method of preventing occurrence of and/or of treating infectious disease in a patient, said method comprising administration, either sequentially or simultaneously, of (i) a recombinant viral vector expressing in vivo at least one heterologous nucleotide sequence, especially an heterologous nucleotide sequence encoding an antigen and (ii) an imidazo[4,5-c]quinolin-4-amine derivative. According to a preferred embodiment, said infectious disease is a viral induced disease, such as for example disease induced by HIV, HCV, HBV, HPV, and the like.

In a further embodiment there is provided the use of an imidazo[4,5-c]quinolin4-amine derivative in the manufacture of a recombinant viral vaccine for the enhancement of an immune response to an antigen encoded by a recombinant viral vector, said recombinant viral vector being administered either sequentially or simultaneously with said derivative.

“Administered sequentially” means that the recombinant viral vector [compound (i)] and the imidazo[4,5-c]quinolin4-amine derivative [compound (ii)] of the present recombinant viral vaccine are administered independently from one another; e.g. a first administration of one of the said compound and a separate second administration consisting in administration of the second compound. According to the present invention, the first administration can be done prior to, concurrently with or subsequent to the second administration, and vice-versa. The therapeutic composition administration and second administration can be performed by different or identical delivery routes (systemic delivery and targeted delivery, or targeted deliveries for example). In a preferred embodiment, each should be done into the same target tissue and most preferably by parenteral route.

In preferred embodiment, the administration of the recombinant viral vector and of the imidazo[4,5-c]quinolin-4-amine derivative is substantially simultaneous. And more preferably, both compounds are co-administered.

In another embodiment, the imidazo[4,5-c]quinolin-4-amine derivative is administered before the administration of the recombinant viral vector. In this special embodiment, “before” means from about 5 min to about 2 weeks, more particularly from about 1 hour to about 1 week, more particularly from about 6 hours to about 48 hours.

The recombinant viral vaccine of the invention is administered in patient as a pharmaceutically acceptable solution, which may routinely contain pharmaceutically acceptable concentrations of salt, buffering agents, preservatives, compatible carriers, adjuvants (e.g. alum, BCG, immune response modifiers), and optionally other therapeutic ingredients.

The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The recombinant viral vaccine can be administered by any ordinary route for administering medications. A variety of administration routes are available. The particular mode selected will depend, of course, upon the particular recombinant viral vaccine content, the particular condition being treated and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed herein. For use in therapy, an effective amount of the 1H-imidazo[4,5-c]quinolin-4-amine derivative can be administered to a subject by any mode that delivers the agent to the desired surface, e.g., mucosal, systemic and under any form, e.g. cream, solution.

The recombinant viral vaccine, or its separate compounds (i) and (ii), may be used according to the invention by a variety of modes of administration, including systemic, topical and localized administration. Injection can be performed by any means, for example by subcutaneous, intradermal, intramuscular, intravenous, intraperitoneal, intratumoral, intravascular, intraarterial injection or by direct injection into an artery (e.g. by hepatic artery infusion) or a vein feeding liver (e.g. injection into the portal vein). Injections can be made with conventional syringes and needles, or any other appropriate devices available in the art. Alternatively the active compound, or any composition containing it, may be administered via a mucosal route, such as the oral/alimentary, nasal, intratracheal, intrapulmonary, intravaginal or intra-rectal route. Topical administration can also be performed using transdermal means (e.g. patch, cream and the like). In the context of the invention, intramuscular and subcutaneous administrations constitute the preferred routes.

For oral administration, the recombinant viral vaccine can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers. Recombinant viral vaccine which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

The recombinant viral vaccine, when it is desirable to deliver it systemically, may be formulated for parenteral administration by injection, e.g., by bolus injection or continuous infusion. Formulations for injection may be presented in unit dosage form, e.g., in ampoules or in multi-dose containers, with an added preservative. The recombinant viral vaccine may take such forms as suspensions, solutions or emulsions in oily or aqueous vehicles, and may contain formulatory agents such as suspending, stabilizing and/or dispersing agents. Recombinant viral vaccine for parenteral administration includes aqueous solutions of the active compounds in water-soluble form. Additionally, suspensions of the active compounds (i) and/or (ii) may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds (i) and/or (ii) may be in powder form for constitution with a suitable vehicle, e.g., sterile pyrogen-free water, before use. The recombinant viral vaccine may also be formulated in rectal or vaginal compositions such as suppositories or retention enemas, e.g., containing conventional suppository bases such as cocoa butter or other glycerides. In addition to the recombinant viral vaccine may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt. The recombinant viral vaccine also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid recombinant viral vaccine forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above.

The 1H-imidazo[4,5-c]quinolin-4-amine derivative may be administered per se or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The recombinant viral vaccine may conveniently be presented in unit dosage form and may be prepared by any of the methods well known in the art of pharmacy. All methods include the step of bringing the compounds (i) and (ii) into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories. For treatment of a patient, depending on activity of the compound, manner of administration, purpose of the immunization (i.e. prophylactic or therapeutic), nature and severity of the disorder, age and body weight of the patient, different doses may be necessary. The administration of a given dose can be carried out both by single administration in the form of an individual dose unit or else several smaller dose units. Multiple administrations of doses at specific intervals of weeks or months apart are usual for boosting the antigen-specific responses.

Other delivery systems can include time-release, delayed release or sustained release delivery systems. Such systems can avoid repeated administrations of the compounds of the recombinant viral vaccine, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

The administration form of the recombinant viral vector [compound (i)] and of the 1H-imidazo[4,5-c]quinolin-4-amine derivative [compound (ii)] can be identical or different for one said recombinant viral vaccine according to the invention (e.g. compound (i) administered as a solution and compound (ii) administered as a cream).

In other aspects, the invention relates to kits. One kit of the invention includes a container containing (i) at least one recombinant viral vector of the invention and a container containing (ii) at least one 1H-imidazo[4,5-c]quinolin-4-amine derivative and instructions for timing of administration of the compounds. The container may be a single container housing both (i) at least one recombinant viral vaccine and (ii) at least one 1H-imidazo[4,5-c]quinolin-4-amine derivative together or it may be multiple containers or chambers housing individual dosages of the compounds (i) and (ii), such as a blister pack. The kit also has instructions for timing of administration of the recombinant viral vaccine. The instructions would direct the subject to take the recombinant viral vaccine at the appropriate time. For instance, the appropriate time for delivery of the recombinant viral vaccine may be as the symptoms occur. Alternatively, the appropriate time for administration of the recombinant viral vaccine may be on a routine schedule such as monthly or yearly. The compounds (i) and (ii) may be administered simultaneously or separately as long as they are administered close enough in time to produce a synergistic immune response.

If desired, the method or use of the invention can be carried out in conjunction with one or more conventional therapeutic modalities (e.g. radiation, chemotherapy and/or surgery). The use of multiple therapeutic approaches provides the patient with a broader based intervention. In one embodiment, the method of the invention can be preceded or followed by a surgical intervention. In another embodiment, it can be preceded or followed by radiotherapy (e.g. gamma radiation). Those skilled in the art can readily formulate appropriate radiation therapy protocols and parameters which can be used (see for example Perez and Brady, 1992, Principles and Practice of Radiation Oncology, 2nd Ed. JB Lippincott Co; using appropriate adaptations and modifications as will be readily apparent to those skilled in the field). In still another embodiment, the method or use of the invention is associated to chemotherapy with one or more drugs (e.g. drugs which are conventionally used for treating or preventing HPV infections, HPV-associated pathologic conditions).

The present Invention further concerns a method for improving the treatment of a cancer patient which is undergoing chemotherapeutic treatment with a chemotherapeutic agent, which comprises co-treatment of said patient along with a recombinant viral vaccine as above disclosed.

The present Invention further concerns a method of improving http://www.micropat.com/perl/di/psrecord.pl?ticket=037405101546&listid=114934200603310905&container_id=763883&patnum=US6015827A cytotoxic effectiveness of cytotoxic drugs or radiotherapy which comprises co-treating a patient in need of such treatment along with a recombinant viral vaccine as above disclosed.

In another embodiment, the method or use of the invention is carried out according to a prime boost therapeutic modality which comprises sequential administration of one or more primer composition(s) and one or more booster composition(s). Typically, the priming and the boosting compositions use different vehicles which comprise or encode at least an antigenic domain in common. The priming composition is initially administered to the host organism and the boosting composition is subsequently administered to the same host organism after a period varying from one day to twelve months. The method of the invention may comprise one to ten sequential administrations of the priming composition followed by one to ten sequential administrations of the boosting composition. Desirably, injection intervals are a matter of one week to six months. Moreover, the priming and boosting compositions can be administered at the same site or at alternative sites by the same route or by different routes of administration. For example, compositions based on HPV early polypeptide can be administered by a mucosal route whereas recombinant viral vaccine is preferably injected, e.g. subcutaneous injection for a MVA vector.

The ability to induce or stimulate an anti-HPV immune response upon administration in an animal or human organism can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed J Wiley & Sons Inc, National Institute of Health). Measurement of cellular immunity can be performed by measurement of cytokine profiles secreted by activated effector cells including those derived from CD4+ and CD8+ T-cells (e.g. quantification of IL-10 or IFN gamma-producing cells by ELIspot), by determination of the activation status of immune effector cells (e.g. T cell proliferation assays by a classical [³H] thymidine uptake), by assaying for antigen-specific T lymphocytes in a sensitized subject (e.g. peptide-specific lysis in a cytotoxicity assay). The ability to stimulate a humoral response may be determined by antibody binding and/or competition in binding (see for example Harlow, 1989, Antibodies, Cold Spring Harbor Press). The method of the invention can also be further validated in animal models challenged with an appropriate tumor-inducing agent (e.g. HPV-E6 and E7-expressing TC1 cells) to determine anti-tumor activity, reflecting an induction or an enhancement of an anti-HPV immune response.

Disease conditions which may especially be treated in accordance with the present invention are for example cervical cancer or precursor lesions of this malignant neoplasia, which are called cervical intraepithelial neoplasia (CIN) or squamous intraepithelial lesions (SIL). The recombinant viral vaccine of the invention may also be useful in the treatment of asymptomatic infections of the cervix in patients identified by DNA diagnosis, or asymptomatic infections that are assumed to remain after surgical treatment of cervical cancer, CIN or SIL, or asymptomatic infections presumed to exist following epidemiological reasoning. The disease conditions to be treated also include genital warts, and common warts and plantar warts. All of these conditions are also caused by a large number of other HPV types, and the agents, compounds and methods of the invention may also be usefully directed against these viruses. All of these lesions presumably derive from asymptomatic infections, that are most often not diagnosed. The present invention may also be usefully targeted against all of these asymptomatic infections.

The invention has been described in an illustrative manner, and it is to be understood that the terminology which has been used is intended to be in the nature of words of description rather than of limitation. Obviously, many modifications and variations of the present invention are possible in light of the above teachings. It is therefore to be understood that within the scope of the appended claims, the invention may be practiced in a different way from what is specifically described herein.

All of the above cited disclosures of patents, publications and database entries are specifically incorporated herein by reference in their entirety to the same extent as if each such individual patent, publication or entry were specifically and individually indicated to be incorporated by reference.

### LEGEND OF THE FIGURES

FIGS. 1a/b/c/d: Therapeutic effect of a combination between topical administration of Imiquimod with subcutaneous injection of MVATG8042. FIG. 1b: Experiment 1: 2.10⁵ TCI sc, 3sc injections with 5.10⁶ pfu, 15 mice per group; FIG. 1c: Experiment 2: 2.10⁵ TCI sc, 3sc injections with 5.10⁶ or 5.10⁵ pfu, 15 mice per group; FIG. 1d: Experiment 3: 2.10⁵ TCI Sc, 3sc injections with 5.10⁶, 15 mice per group

FIGS. 2a/2b: Measure of the frequence of E7/E6 specific IFNγ secreting lymphocytes. FIG. 2b: E6/E7 specific INFgamma/Elispot Th1 response.

FIG. 3: IL-4 ELISPOT assay. E7 specific IL-4/Elispot Th2 response.

FIG. 4a/4b: Flow cytometry analysis of R9F-specific CD8+ T cells. Measure of the frequence of Tet_R9F⁺ E7 specific CD8⁺ T cells.

FIG. 5a/5b: E7-specific humoral immune response. Measure of Th1/Th2 isotype IgG switch.

FIG. 6: MVA-specific neutralizing antibody titer (NAT50).

FIG. 7: Therapeutic effect of Aldara+MVATG9931 combination in a RenCa-Muc1 tumour model.

FIG. 8: Effect of imiquimod on MUC1-specific Th1 type T cell responses against short or long epitopes.

FIG. 9: IL-4 elispot assay: Effect of imiquimod on MUC-1 specific Th2 type T cell responses.

FIG. 10: MUC1 specific humoral immune response (Isotype Switch): Effect of imiquimod on MUC 1 specific humoral response.

### EXAMPLES

A—Recombinant Viral Vector Expressing HPV Antigens.

1. Materials and Methods

1.1. Test Article

Denomination and Brief description of each Recombinant Cector Construction (Based on MVA)

| | Batch | E6tm/ | |
|---|---|---|---|
| Virus | concentration | E7tm | hIL-2 |
| Denomination | (pfu/ml) | promoter | promoter |
| MVAN33 | 4.5 · 10⁹ pfu/ml | — | — |
| MVATG8042 | 3.1 · 10⁹ pfu/ml | P7.5 | PH5R |

### Conditions of Storage:

Viruses were maintained at −80° C. until the day of injection. The viral suspension was rapidly thawed immediately prior to dilution and administration.

Viruses were diluted in buffer Tris/HCl 10 mM, saccharose 5% (w/v), 10 mM NaGlu, 50 mM NaCl, pH8.0 in order to obtain the required dose in a 100 μl volume.

1.2. Animal Model

Species/Strain/Supplier:

SPF healthy female C57Bl/6 mice were obtained from Charles River (Les Oncins, France).

The animals were 6-weeks-old upon arrival. At the beginning of experimentation, they were 7-week-old.

The animals were housed in a single, exclusive room, air-conditioned to provide a minimum of 11 air changes per hour. The temperature and relative humidity ranges were within 20° C. and 24° C. and 40 to 70% respectively. Lighting was controlled automatically to give a cycle of 12 hours of light and 12 hours of darkness. Specific pathogen free status was checked by regular control of sentinel animals.

Throughout the study the animals had access ad libitum to sterilized diet type RM1 (Dietex France, Saint Gratien). Sterile water was provided ad libitum via bottles.

All animals were acclimatized for one week before the start of the experiment.

1.3. Cells Description

TC1 tumour cells obtained from C57Bl6 mice lung, have been transduced with two retroviruses: LXSN16E6E7 expressing E6 and E7 from HPV16 and pVEJB expressing the ras gene. The cells were cultured in DMEM containing 0.5 mg/ml G418 and 0.2 mg/ml Hygromycine. Adherents cells were removed by trypsine treatment and after 3 washings, tumour challenge were performed subcutaneously with 2.10⁵ TC1 viable cells.

1.4. Aldara™ (3M Pharmaceuticals)

Aldara™ is the brand name for imiquimod. Each gram of the 5% cream contains 50 mg of imiquimod in an off-white oil-in-water vanishing cream base consisting of isostearic acid, cetyl alcohol, stearyl alcohol, white petrolatum, polysorbate 60, sorbitan monostearate, glycerine, xanthan gum, purified water, benzyl alcohol, methylparaben and propylparaben.

1.5. Protocol

Immunizations Schedule:

For the immunotherapeutic experiments, 15 C57Bl6 female mice were challenged subcutaneously in the right flank with 2.10⁵ TC1 cells at D1. Mice were treated three times, subcutaneously at three distant sites, with 5.10⁶ pfu or 5.10⁵ pfu of vaccinia virus at D8, D15 and D22. Imiquimod (Aldara 5% cream; 3M Pharmaceuticals) was applied topically just before each immunization over the sites of injection to the shaved skin of mice (approx. 1 cm²). Each mouse received approximately 0.8 mg or 1.6 mg/mouse of active imiquimod per immunization. Tumour growth was monitored, twice a week during 80 days, with a calliper. Mice were euthanized for ethical reasons when their tumour size was superior to 25 mm of diameter or when they showed pain even if the tumour was smaller.

For the immunogenicity study, 3 tumor-free C57Bl6 female mice were vaccinated subcutaneously at three distant sites with 5.10⁷ pfu or 5.10⁶ pfu of vaccinia virus at D1, D8 and D15. This dose was used to optimize the detection of cellular immunity against HPV specific antigens. Imiquimod was applied topically just before each immunization over the sites of injection to the shaved skin of mice (approx. 1 cm²). Each mouse received approximately 0.8 mg or 1.6 mg/mouse of active imiquimod per immunization. Spleen and serum were removed at D22 for immunological analysis.

Parameters of Monitoring:

*Measure of the Number/Frequency of IFNgamma (Th1) or IL-4 (Th2) Secreting Cells by Elispot

Fresh spleen cells were prepared using a Cell Strainer (BD Falcon). All the peptides were synthesized by Neosystem at the immunograde level (10 mg). Each peptide was dissolved in DMSO at 10 mg/ml and store at 4° C. Elispot was carried out using the Mabtech AB mouse IFNgamma ELISPOT^{PLUS} kit or mouse IL-4 ELISPOT^{PLUS} kit (Mabtech, France) according to the manufacturer's instructions. A 96-well nitrocellulose plate was coated with 3 μg/ml monoclonal rat anti-mouse IFNgamma antibody (Clone R4-6A2; Pharmingen, cat. nr551216, Lot M072862; 100 μl/well) in Sodium Carbonate Buffer. The plates were incubated overnight at 4° C. or 1 h at 37° C. Plates were washed three times with DMEM 10% FCS and saturated 2 hours at 37° C. with 100 μl DMEM 10% FCS/well. Splenocytes were plated at a concentration of 10⁶ cells/100 μl. Interleukine 2 was added to all the wells at a concentration of 6U/50 μl/well (R&D Systems) 10 ng/ml). ConcanavalinA was used as positive control (5 μg/ml). HPV specific peptides were used at a concentration of 5 μg/ml. The plates were incubated 48 hours at 37° C., 5% CO2. The plate was washed one time with PBS 1× and 5 times with PBS-Tween 0.05%. Biotinylated Anti-mouse IFNgamma (clone XMG1.2, Pharmingen) was added at the concentration of 0.3 μg/100 μl/well and incubated 2 hours at room temperature under slow agitation. The plate was washed 5 times with PBS-Tween 0.05%. Extravidin AKP (Sigma, St. Louis, Mo.) diluted at 1/5000 in PBS-Tween 0.05%-FCS1% was also added to the wells (100 μl/well). The plate was incubated 45 minutes at room temperature and then washed 5 times with PBS-Tween 0.05%. IFNgamma secretion was revealed with Biorad Kit. 100 μl substrate (NBT+BCIP) was added per well and plate was left at room temperature for ½ hour. The plate was washed with water and put to dry overnight at room temperature. Spots were counted using a dissecting microscope. Spots were counted using the Elispot reader Bioreader 4000 Pro-X (BIOSYS-Gmbh; Serlabo France).

List of Tested Peptides:

| | |
|---|---|
| SCVYCKKEL (E6; Db) | S9L Peptide |
| RCIICQRPL (E6; Db) | R9L Peptide |
| SEYRHYQYS (E6; Kb) | S9S Peptide |
| ECVYCKQQL (E6; Db) | E9L Peptide |
| TDLHCYEQL (E7; Kb) | T9L Peptide |
| RAHYNIVTF (E7; Db) | R9F Peptide |

Irrelevant Peptide (MUC1 Specific)

D38L (E7; Db) is a 38 amino acid-long E7-specific peptide. Recombinant purified E7 protein has also been used in the different ELISPOT assays.

*Measure of the Frequency of R9F Tetramer Specific CD8⁺ T Cells

Fresh spleen cells were harvested and prepared using a BD specific sieve (Cell Strainer). Splenocytes were stimulated during 5 days with R9F peptide (5 μg/ml) in 24 well plates or used directly for specific labelling. 1.10⁶ cells were stained with 1 μl of an APC-coupled mouse CD8 specific antibody (BD Pharmingen 553035; clone 53-6.7; lot no 32567) and 10 μl of R9F specific H-2Db tetramer (Beckman Coulter T20071; H-2Db/PE; peptide RAHYNIVTF; lot C507117; C602110) during 30 min at 4° C. Cells were washed then diluted in PBS/0.5% PFA.

*Measure of Th1/Th2 Related IgG Isotype Switch Against E7 Antigens

*96-well plates were coated overnight at 4° C. with 3 μg/ml of E7 purified protein (P#2101 cahier PC00001; page 157; October 2002).The protein was diluted in coating buffer (200 mM NaHCO₃, 80 mM Na₂CO₃, pH 9.5) and 100 μl were added per well.

* Wells were washed five times with a plate washer (PBS, 0.1% Tween 20, 10 mM EDTA) and saturated for 1 hour at room temperature with 300 μl PBS+3% BSA.

* Wells were washed 5 times and incubated with ½ serial dilutions of mouse serum (1/25 to 1/1600 in PBS+1% BSA) for 2 hours at room temperature.

* The plate was washed 5 times. A peroxidase-conjugated rat anti-mouse IgG2a (BD Pharmingen 553391) or a rat anti-mouse IgG1 (BD Pharmingen 559626) diluted at 1/1000 in PBS+1% BSA was added (100 μl/well) and incubated for 1 hour at room temperature.

*Wells were washed five times and revealed with 100 μl substrate solution (0.05M citric acid, 0.05M sodium acetate, 1% tetramethylbenzidine, 0.015% H₂O₂)/well.

TMB solution (10 ml)=140 μl TMB+2 μl H₂O₂+5 ml Na Acetate (0.1M)+5 ml Citrate (0.1M)

*The reaction was stopped by adding 100 μl of 0.8M H₂SO₄/well. Absorbance was measured at 450 nm (Genesys system).

*Analysis of MVA-Neutralizing Antibody Induced by the Vaccination

Cells: BHK-21 (hamster fibroblast, Ref ATCC: CCL-10)

MVA-GFP (MVATG15938): Reporter green fluorescent protein was inserted in MVA deletion III under the control of p11K7.5 promoter.

Step 1: Neutralizing Serum:

All sera were decomplemented by heating for 30 min at 56° C. before use.

Positive control: serum from rabbit immunized with

Poxvirus (WR strain) (Ref. Ac WR IMVQC34)

Step 2: Seroneutralization Assay (SOP Measurement of Neutralizing Anti-MVA Antibodies Titer)
Plasma were serially diluted in culture medium (range of dilution 50× to 3200×) and incubated in a 96-well microplate with MVA-GFP (5×10³ pfu/well) for 1 h at 37° C. (neutralization step). BHK-21 cells (10⁵ cells/well) were then seeded and incubated for an additional 16-18 hours at 37° C., 5% CO₂.

The next day, the 96-well microplate was washed with 250 μL of PBS and 100 μL of PBS was added to each well before reading fluorescence intensity with a Fluorescence Microplate reader (VICTOR™ PerkinElmer®). The neutralizing antibody titre is the titre at which 50% viral activity is inhibited. Neutralizing Antibody Titres (NAT₅₀) were calculated using the Spearman-Kärber method.

2—Results

Three independent therapeutic experiments have been performed following the model described in the Material and Method section.

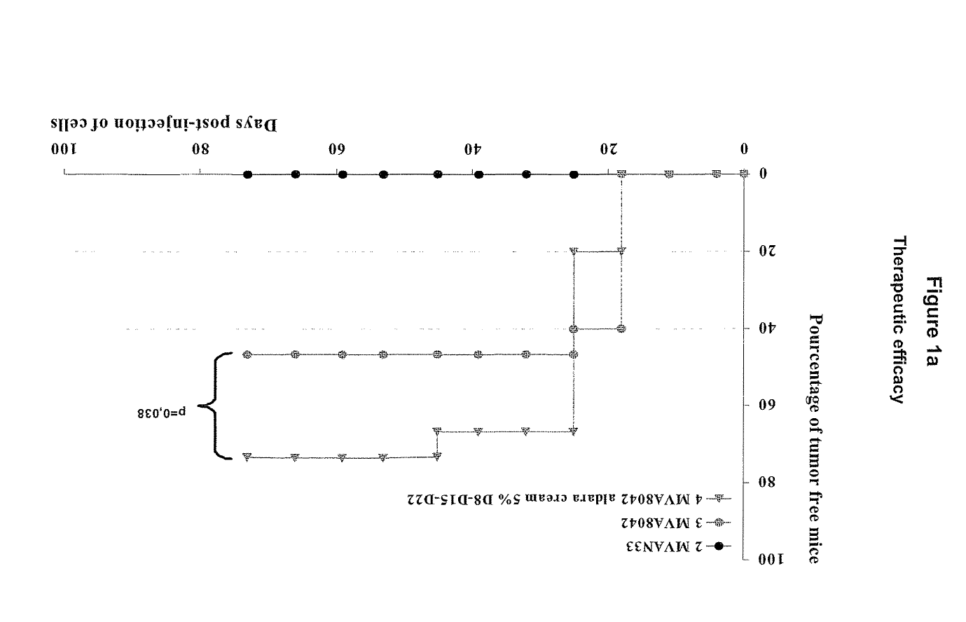

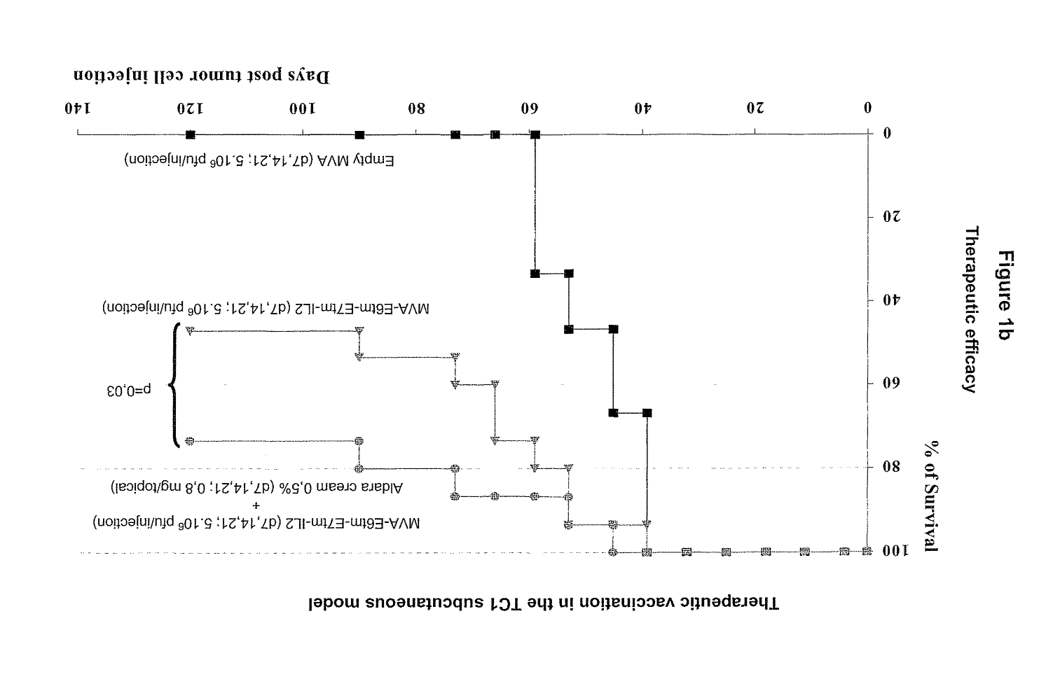

In all experiments, we have consistently observed that topical application of Aldara™ cream (5%) at the site of vaccination increases significantly the therapeutic efficacy of MVATG8042 (see FIG. 1a, b, c and d). In this setting, vaccination with 5.10⁶ pfu of MVATG8042 induced on average 45% tumor-free mice by the end of each experiment while 75% and 95% tumor-free animals were seen when MVATG8042 was used in combination with 0.8 mg or 1.6 mg imiquimod, respectively.

In one experiment series (see FIG. 1c), we have also observed that the addition of Aldara™ allows reaching the same therapeutic efficacy with a one log lower dose of virus (5.10⁵ pfu).

The statistical difference in the in vivo survival experiments between the different groups was assessed using a Log Rank application (Statistica 5.1 software, Statsoft Inc.) of the Kaplan-Meier survival curves. A p≦0.05 is considered statistically significant.

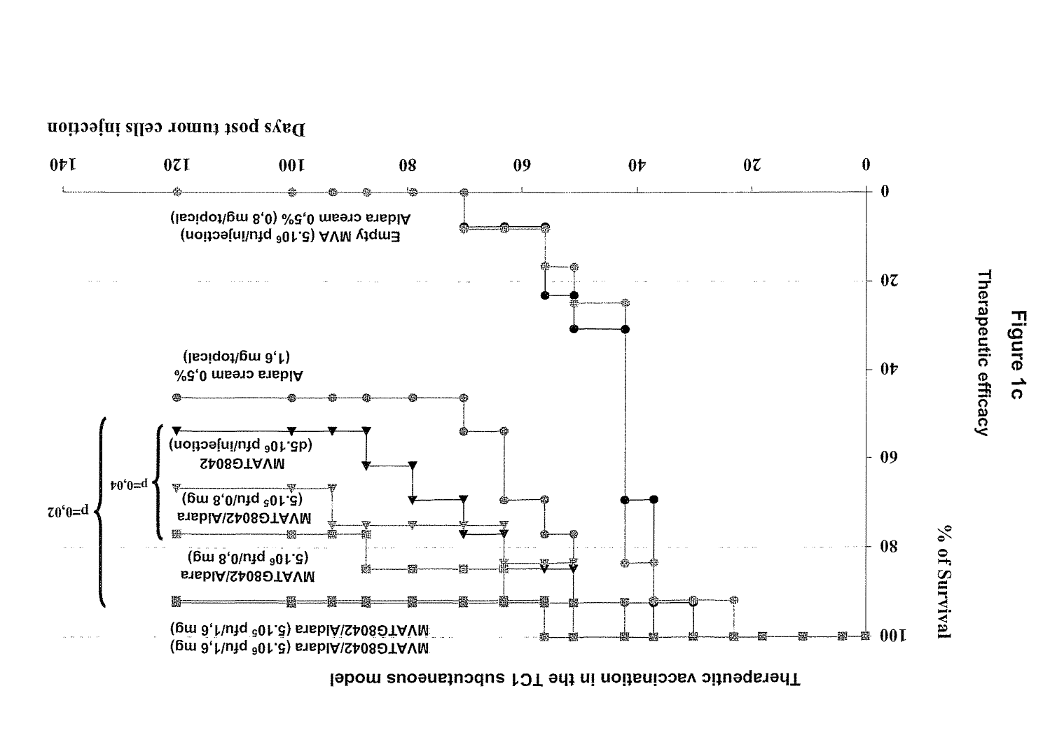

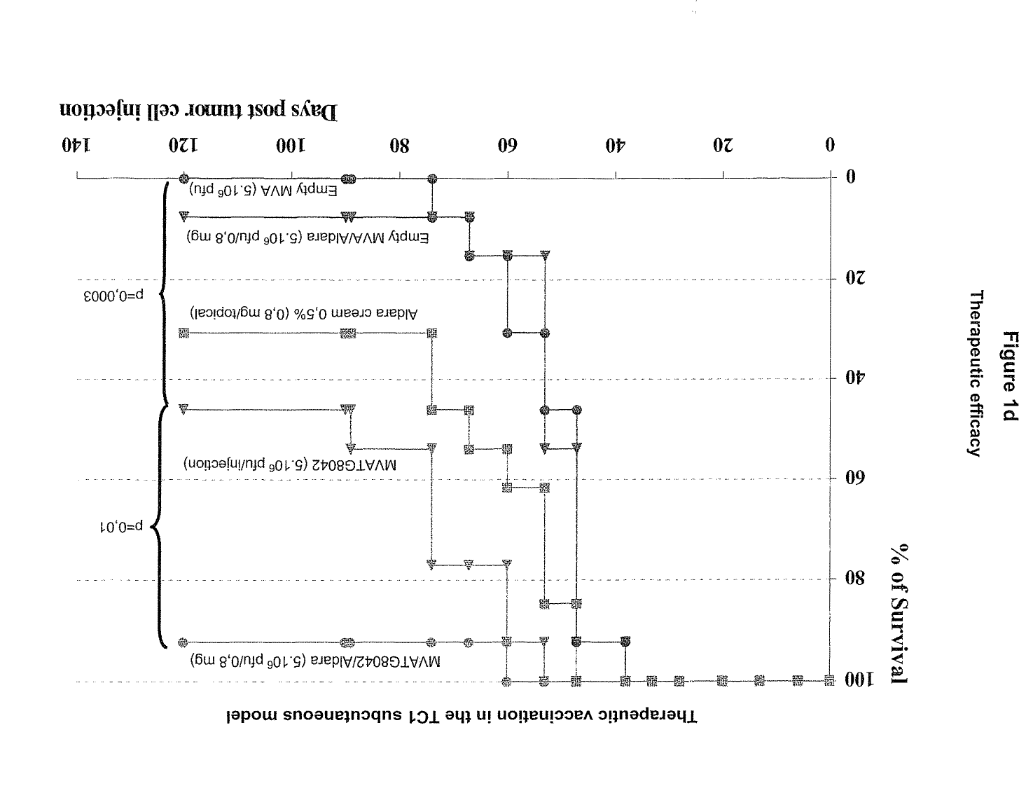

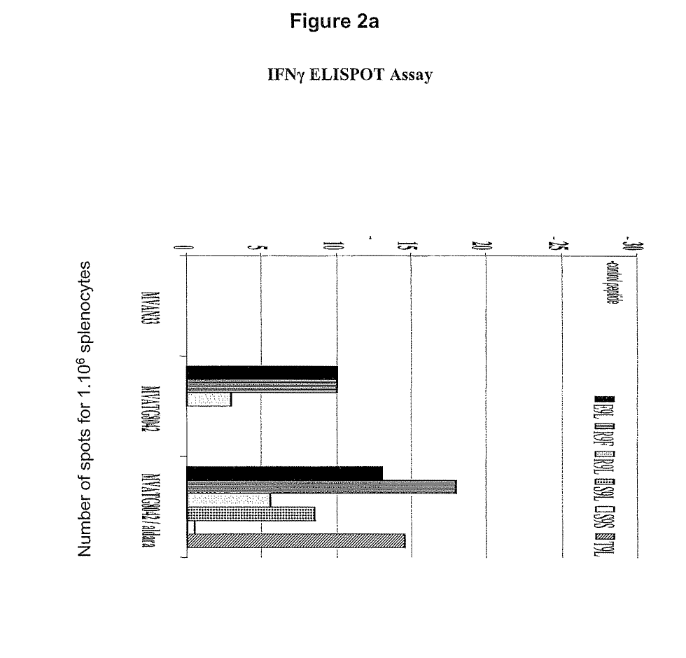

In parallel, two independent studies were performed to evaluate the induction of both cellular and humoral responses against E6 and E7 HPV antigens. Mice were vaccinated as described in the protocol section. In both experiments, the number of E6 or E7-specific IFNgamma secreting cells was enumerated using an ELISPOT assay. These results show that topical administration of Aldara™ results in a significant increase in the number of MHC class I restricted CD8+ T cells relative to the one obtained with MVATG8042 alone (FIGS. 2a and b). Low responses to a broader range of epitopes are present in the Aldara™+MVATG8042 group (peptide S9S and T9L). In parallel, in a separate experiment, the number of E7-specific IL-4 secreting cells was lower in the MVATG8042+Aldara™ than in the MVA alone group (FIG. 3). Taken together, these data indicate that the combination of MVATG8042+Aldara™ improves the Th1-based cellular immune response against E6 and E7 antigens.

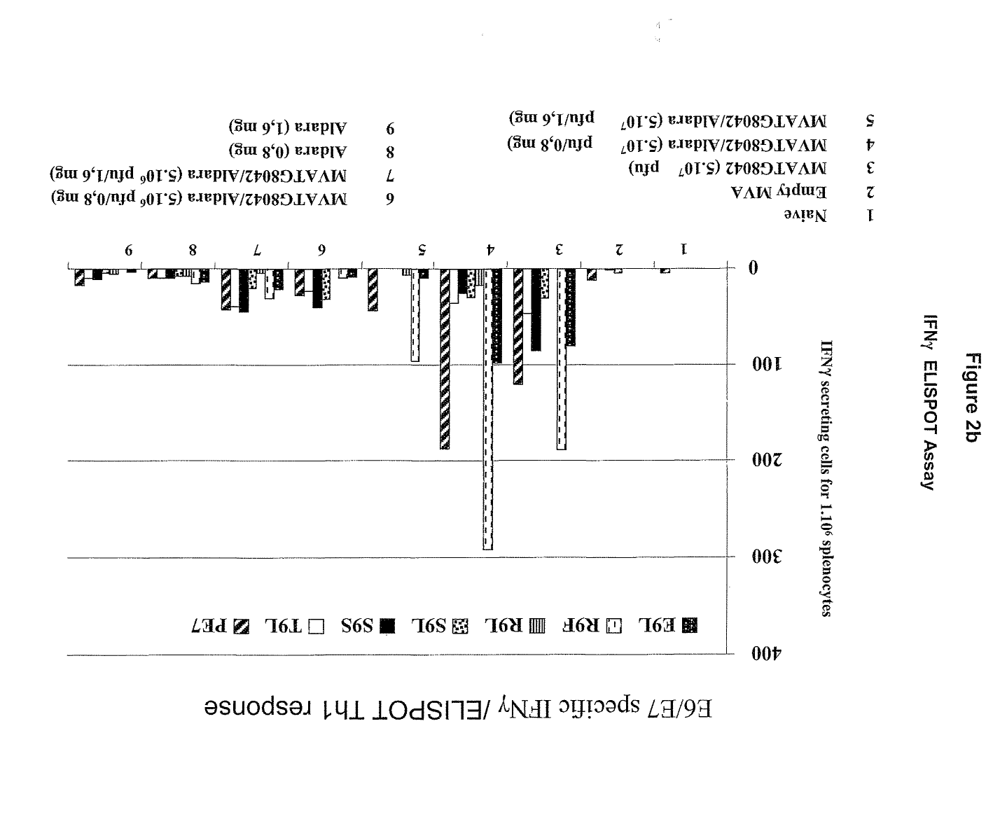

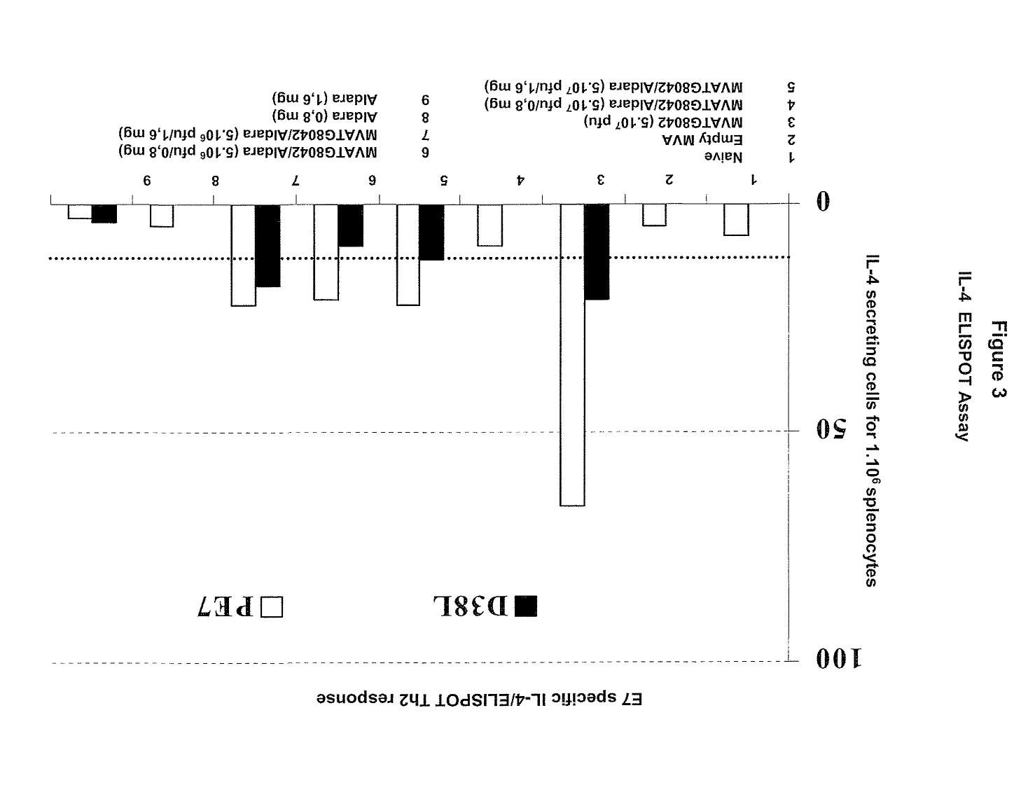

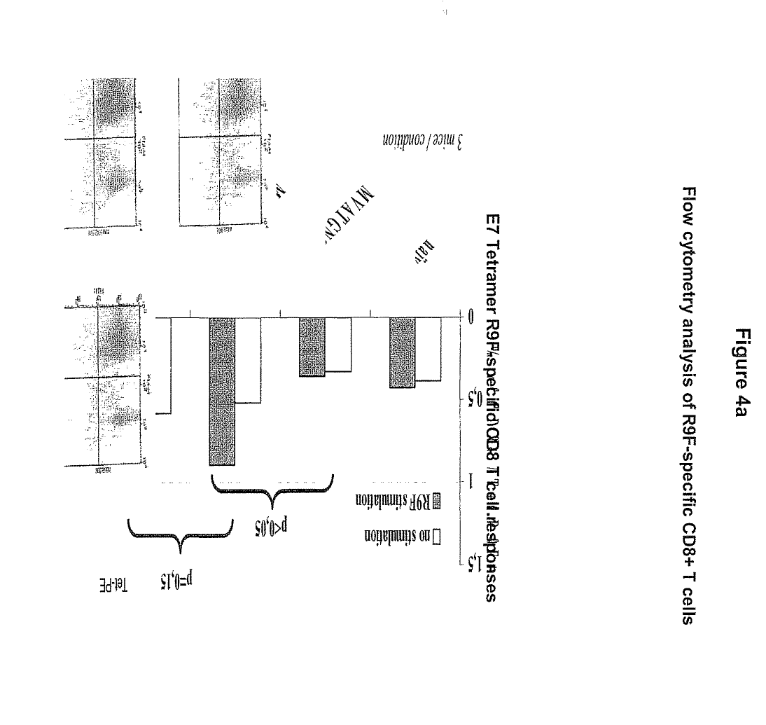

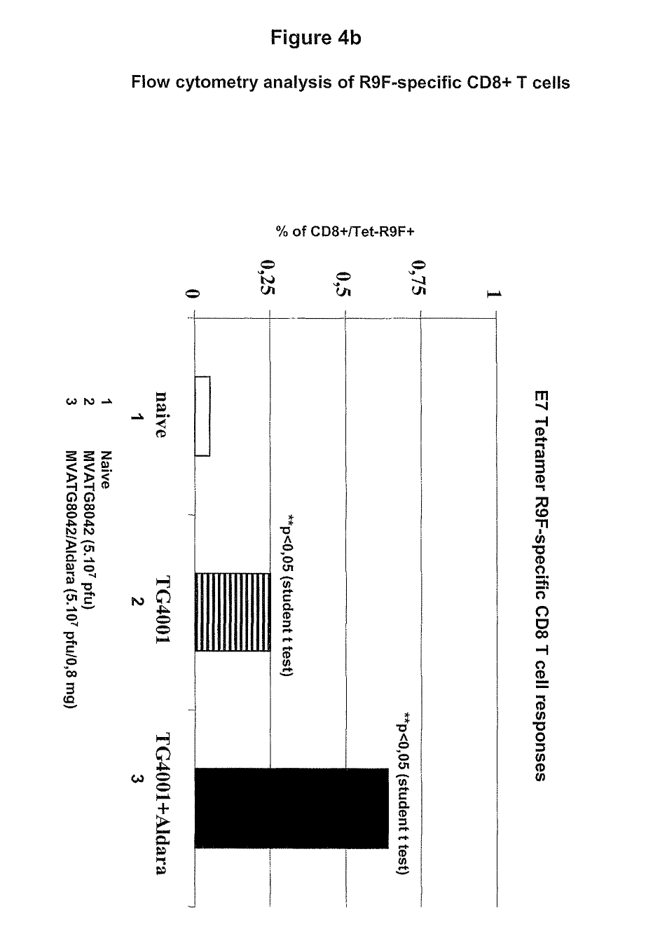

The frequency of CD8⁺/R9F Tetramer⁺ splenocytes has further been analysed by flow cytometry before or after in vitro stimulation with the E7-specific immunodominant epitope R9F (FIG. 4a). These results indicate that recognition of the R9F immunodominant epitope is clearly mediated by CD8⁺ specific T cells. The frequency of the R9F-Db-restricted CD8+ population is low in the spleen and this population is better detected after an in vitro stimulation with the peptide. Pre-treatment with Aldara™ improved significantly the number of R9F-Db-specific CD8⁺ T cells in experiment shown in FIG. 4b.

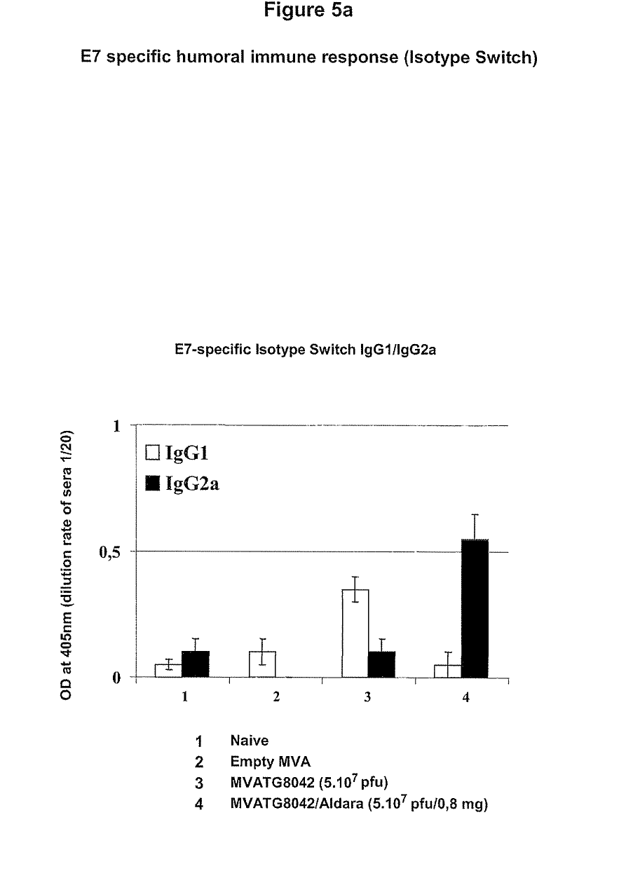

Finally the measure of the humoral response against the E7 antigen was also performed by ELISA. In order to better characterize the type of response induced by the combination Aldara™+MVATG8042, the IgG isotype switch was analysed. E7-specific IgG1 and IgG2a were detected. The data (FIG. 5) show that topical application of Aldara™ induces a typical Th1 profile (higher IgG2a titer than IgG1, FIG. 5a) which could have implications in the efficacy of the combined treatment. These results are confirmed in a second experiment (FIG. 5b).

Finally the level of MVA-specific neutralizing antibody was measured to analyse the impact of Aldara combination with MVATG8042 (FIG. 6). The results indicate that combining MVATG8042 and Aldara™ reduces the titer of MVA-specific neutralizing antibody obtained when compared to similarly injected MVA alone. This could be explained by the environment created by the topical administration of the Aldara™ cream which may protect against the neutralization by specific antibodies.

B—Recombinant Viral Vector Expressing Tumoral Antigen MUC1.

2.1.Test Article

### Denomination and Brief Description of Each Recombinant Vector Construction (Based on MVA)

| | | Batch | |
|---|---|---|---|
| Virus | | concentration | Encoded |
| Denomination | Batch Number | (pfu/ml) | genes |
| MVAN33 | P925PB | 2.4 · 10⁹ pfu/ml | — |
| MVATG9931 | P920W1 | 1.5 · 10⁹ pfu/ml | MUC1; hIL-2 |

Conditions of Storage:

Viruses were received from the Molecular Immunology Department and then were maintained at −80° C. until the day of injection. The viral suspension was rapidly thawed immediately prior to dilution and administration.

Conditions of Dilution Before Use:

Viruses were diluted in TG0008 buffer (Tris/HCl 10 mM, saccharose 5% (w/v), 10 mM NaGlu, 50 mM NaCl, pH8.0) in order to obtain the required dose in a 100 μl volume.

2.2. Animal Model

Species/Strain/Supplier:

SPF healthy female B6D2 and C57Bl/6 mice were obtained from Charles River (Les Oncins, France).

The animals were 6-weeks-old upon arrival. At the beginning of experimentation, they were 7-week-old. The animals were housed in a single, exclusive room, air-conditioned to provide a minimum of 11 air changes per hour. The temperature and relative humidity ranges were within 20° C. and 24° C. and 40 to 70% respectively. Lighting was controlled automatically to give a cycle of 12 hours of light and 12 hours of darkness. Throughout the study the animals had access ad libitum to sterilized diet type RM1 (Dietex France, Saint Gratien). Sterile water was provided ad libitum via bottles.

2.3. Cells Description

RenCa-MUC1 tumor cells: RenCa is an experimental murine kidney cancer model. RenCa cells were transfected with pHMG-ETAtm (MUC-1) and pY3 (hygromycin B resistance) using the classical Ca²⁺ phosphate transfection method. Clones were selected after clonal dilution in DMEM (Dulbecco Modified) supplemented with 10% inactivated fetal calf serum, L-glutamin (2 mM), gentamycin (0.04 g/l) and hygromycin (600 μg/ml, Roche Diagnostic). Analysis of MUC-1 expression was made by cytofluorimetry analysis (using a FACScan, Becton Dickinson) with H23 monoclonal antibody. Adherents cells were removed by PBS/EDTA treatment and after 3 washings, tumor challenge were performed subcutaneously with 3.10⁵ RenCa-MUC1 (clone 4) viable cells.

2.4. Protocol

Immunizations Schedule:

For the immunotherapeutic experiments, 15 B6D2 female mice were challenged subcutaneously in the right flank with 3.10⁵ RenCa-MUC1 cells at D1. Mice were treated three times, subcutaneously at three distant sites, with 5.10⁷ pfu of poxvirus (MVA strain) at D4, 11 and 18. Imiquimod was applied topically just before each immunization over the sites of injection to the shaved skin of mice (approx. 10 cm²). Each mouse received approximately 1 mg/mouse of active imiquimod per immunization. Tumour growth was monitored, twice a week during 80 days, with a calliper. Mice were euthanized for ethical reasons when their tumour size was superior to 25 mm of diameter or when they showed pain even if the tumour was smaller.

For the immunogenicity study, 3 C57Bl6 female mice were vaccinated subcutaneously at three distant sites with 5.10⁷ pfu of poxvirus (MVA strain) at D1, 8 and 15. This dose was used to optimize the detection of cellular immunity against MUC1 specific antigens. Imiquimod was applied topically just before each immunization over the sites of injection to the shaved skin of mice (approx. 10 cm²). Each mouse received approximately 1 mg/mouse of active imiquimod per immunization. Spleen and serum were removed at D22 for immunological analysis.

Parameters of Monitoring:

*Measure of the Number/Frequency of IFNgamma Secreting Cells by Elispot

Fresh spleen cells were prepared using Lympholite purification buffer. All the peptides were synthesized by Neosystem at the immunograde level (10 mg). Each peptide was dissolved in DMSO at 10 mg/ml and store at 4° C. A 96-well nitrocellulose plate was coated with 3 μg/ml monoclonal rat anti-mouse IFNgamma antibody (Clone R4-6A2; Pharmingen, cat. nr551216, Lot M072862; 100 μl well) in Sodium Carbonate Buffer. The plates were incubated overnight at 4° C. or 1 h at 37° C. Plates were washed three times with DMEM 10% FCS and saturated hours at 37° C. with 100 μl DMEM 10% FCS/well. Splenocytes were plated at a concentration of 10⁶ cells/100 μl. Interleukine was added to all the wells at a concentration of 6U/50 μl/well (R&D Systems) 10 ng/ml). ConcanavalinA was used as positive control (5 μg/ml). MUC1 specific peptides were used at a concentration of 5 μg/ml. The plates were incubated 48 hours at 37° C., 5% CO2. The plate was washed one time with PBS 1× and 5 times with PBS-Tween 0.05%. Biotinylated Anti-mouse IFNgamma (clone XMG1.2, Pharmingen) was added at the concentration of 0.3 μg/100 μl/well and incubated 2 hours at room temperature under slow agitation. The plate was washed 5 times with PBS-Tween 0.05%. Extravidin AKP (Sigma, St. Louis, Mo.) diluted at 1/5000 in PBS-Tween 0.05%-FCS1% was also added to the wells (100 μl/well). The plate was incubated 45 minutes at room temperature and then washed 5 times with PBS-Tween 0.05%. IFNgamma secretion was revealed with Biorad Kit. 100 μl substrate (NBT+BCIP) was added per well and plate was left at room temperature for ½ hour. The plate was washed with water and put to dry overnight at room temperature. Spots were counted using a dissecting microscope.

*Measure of the Number/Frequency of IL-4 Secreting Cells by Elispot

Fresh spleen cells were prepared using Lympholite purification buffer. All the peptides were synthesized by Neosystem at the immunograde level (10 mg). Each peptide was dissolved in DMSO at 10 mg/ml and, store at 4° C. A 96-well nitrocellulose plate was coated with 3 μg/ml monoclonal anti-mouse IL-4 antibody (Pharmingen, cat. nr551878, Lot 27401; 100 μl/well) in Sodium Carbonate Buffer. The plates were incubated overnight at 4° C. or 1 h at 37° C. Plates were washed three times with DMEM 10% FCS and saturated 2 hours at 37° C. with 100 μl DMEM 10% FCS/well. Splenocytes were plated at a concentration of 10⁶ cells/100 μl. Interleukine 2 was added to all the wells at a concentration of 6U/50 μl/well (R&D Systems 10 ng/ml). ConcanavalinA was used as positive control (5 μg/ml). MUC1 specific peptides were used at a concentration of 5 μg/ml. The plates were incubated 48 hours at 37° C., 5% CO2. The plate was washed one time with PBS 1× and 5 times with PBS-Tween 0.05%. Biotinylated Anti-mouse IL-4 (Pharmingen) was added at the concentration of 0.2 μg/100 μl/well and incubated 2 hours at room temperature under slow agitation. The plate was washed 5 times with PBS-Tween 0.05%. Extravidin AKP (Sigma, St. Louis, Mo.) diluted at 1/5000 in PBS-Tween 0.05%-FCS1% was also added to the wells (100 μl/well). The plate was incubated 45 minutes at room temperature and then washed 5 times with PBS-Tween 0.05%. IFNgamma secretion was revealed with Biorad Kit. 100 μl substrate (NBT+BCIP) was added per well and plate was left at room temperature for ½ hour. The plate was washed with water and put to dry overnight at room temperature. Spots were counted using a dissecting microscope.

List of Tested Peptides:

| | |
|---|---|
| F9L | FLSFHISNL (H-2K^{b}; Heukamp, 2001) |
| A9A | APGSTAPPA (H-2D^{b}) |
| T24P | TAPPAHGVTSAPDTRPARGSTAPP |
| G23D | GQDVTLAPATEPASGSAATWGQD |
| V23S | VTGSGHASSTPGGEKETSATQRS |

Irrelevant Peptide/R9F RAHYNIVTF (E7; Db)

*Measure of Th1/Th2 Related IgG Isotype Switch Against MUC1 Antigen

*96-well plates were coated overnight at 4° C. with 3 μg/ml of T24P MUC1 specific peptide. The peptide was diluted in coating buffer (200 mM NaHCO₃, 80 mM Na₂CO₃, pH 9.5) and 100 μl were added per well.

*Wells were washed five times with a plate washer (PBS, 0.1% Tween 20, 10 mM EDTA) and saturated for 1 hour at room temperature with 300 μl PBS+3% BSA.

*Wells were washed 5 times and incubated with ½ serial dilutions of mouse serum (1/25 to 1/1600 in PBS+1% BSA) for 2 hours at room temperature.

*The plate was washed 5 times. A peroxidase-conjugated rat anti-mouse IgG2a (BD Pharmingen 553391) or a rat anti-mouse IgG1 (BD Pharmingen 559626) diluted at 1/1000 in PBS+1% BSA was added (100 μl/well) and incubated for 1 hour at room temperature.

*Wells were washed five times and revealed with 100 μl substrate solution (0.05M citric acid, 0.05M sodium acetate, 1% tetramethylbenzidine, 0.015% H₂O₂)/well.

TMB solution (10 ml)=140 μl TMB+2 μl H₂O₂+5 ml Na Acetate (0.1M)+5 ml Citrate (0.1M)

*The reaction was stopped by adding 100 μl of 0.8M H₂SO₄/well. Absorbance was measured at 450 nm (Genesys system).

3—Results

A therapeutic experiment has been done in the RenCa-MUC1 subcutaneous model as described in the protocol section. We have observed that a pre-treatment by a topical administration of Aldara™ cream 5% increase significantly the therapeutic efficacy of MVATG9931 by 5% to 35% of tumor free mice at the end of the experiment. In this experiment, no mice were treated with topical application of Aldara™ only. However, according to published information on a different cancer model (OVA expressing tumor), it is described that topical application of Aldara™ at a distinct site than the tumor has no therapeutic effect (Craft et al., 2005). The statistical difference in in vivo survival experiment between the different groups was assessed using a Log Rank application (Statistica 5.1 software, Statsoft Inc.) of the Kaplan-Meier survival curves. A P≦0.05 is considered statistically significant.

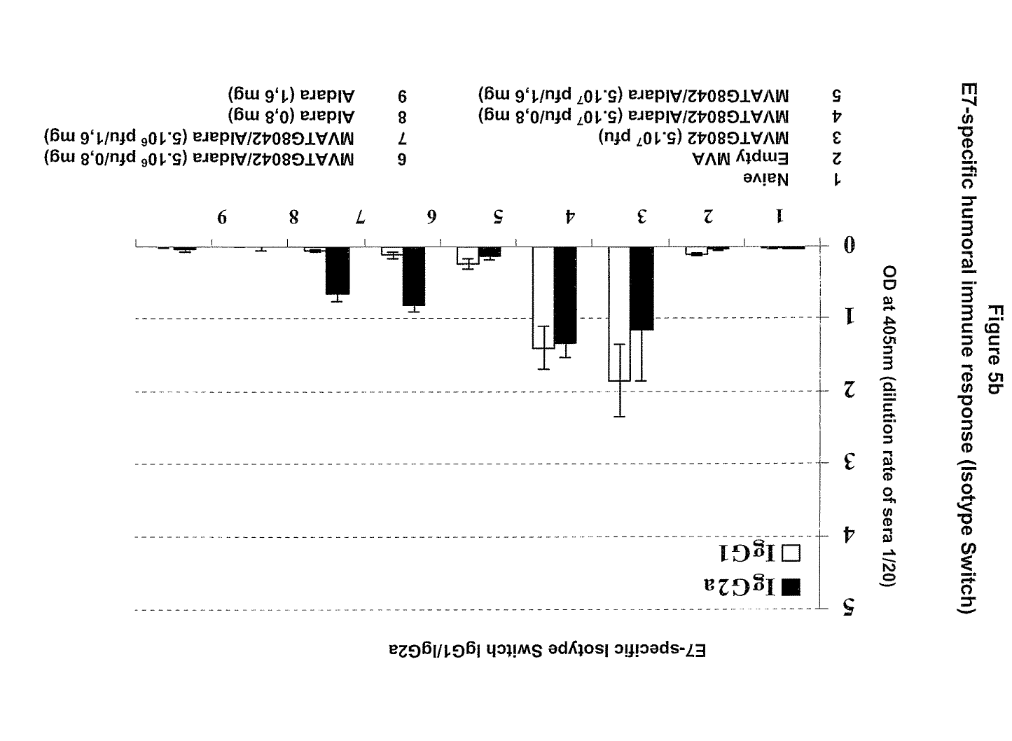

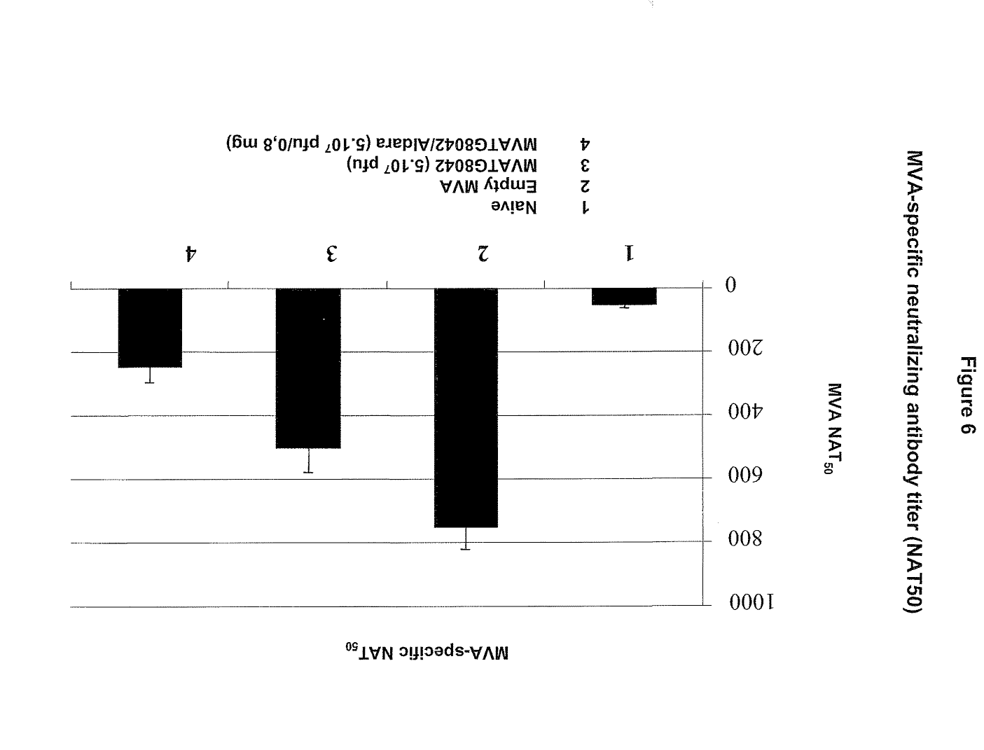

FIG. 7 illustrates the therapeutic effect of Aldara+MVATG9931 combination in a renCa-Muc1 tumour model.

An immunogenicity study was also performed in parallel to look for the induction of both cellular and humoral responses against MUC1 antigen. Mice were vaccinated as described in the protocol section.

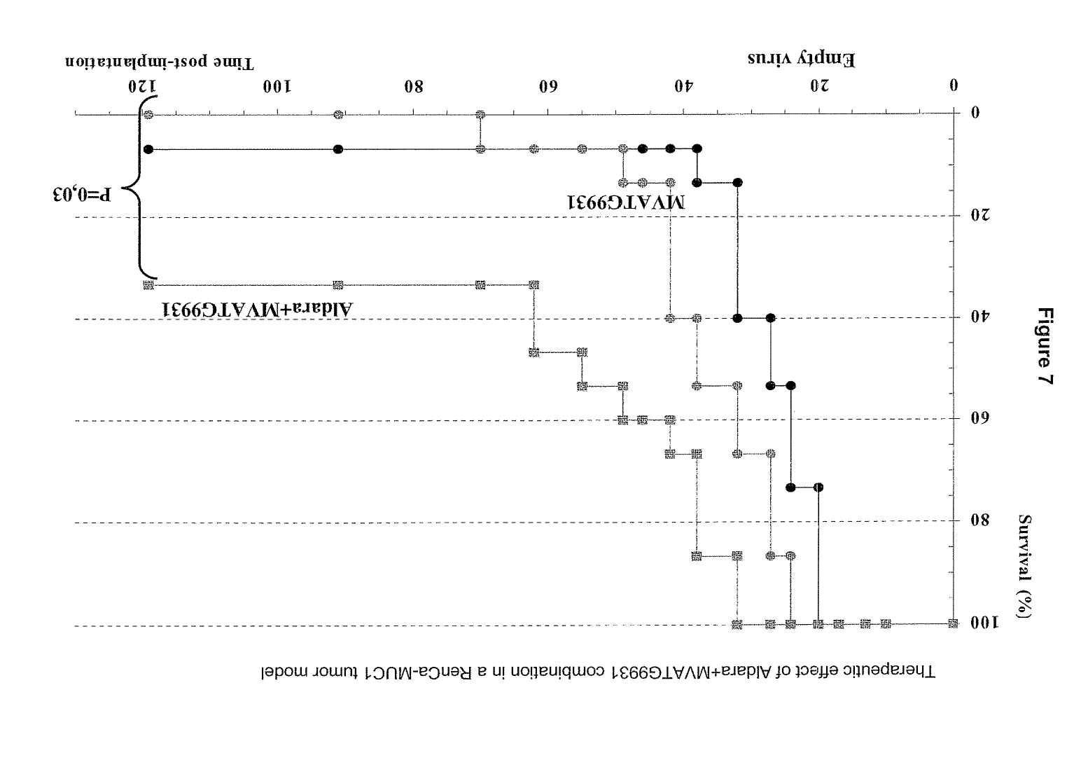

In a first set of experiments, the number of MUC1-specific IFNgamma secreting cells was enumerated using an ELISPOT assay. MUC1 H-2D^{b}, H-2K^{b} and long restricted peptides were used to monitored both CD4 and CD8 T cell response after immunization. We have observed that pre-treatment with a topical administration of Aldara does not improve significantly the number of MHC class I and class II restricted CD4 and CD8 T cells obtained with MVATG9931 alone (FIG. 8).

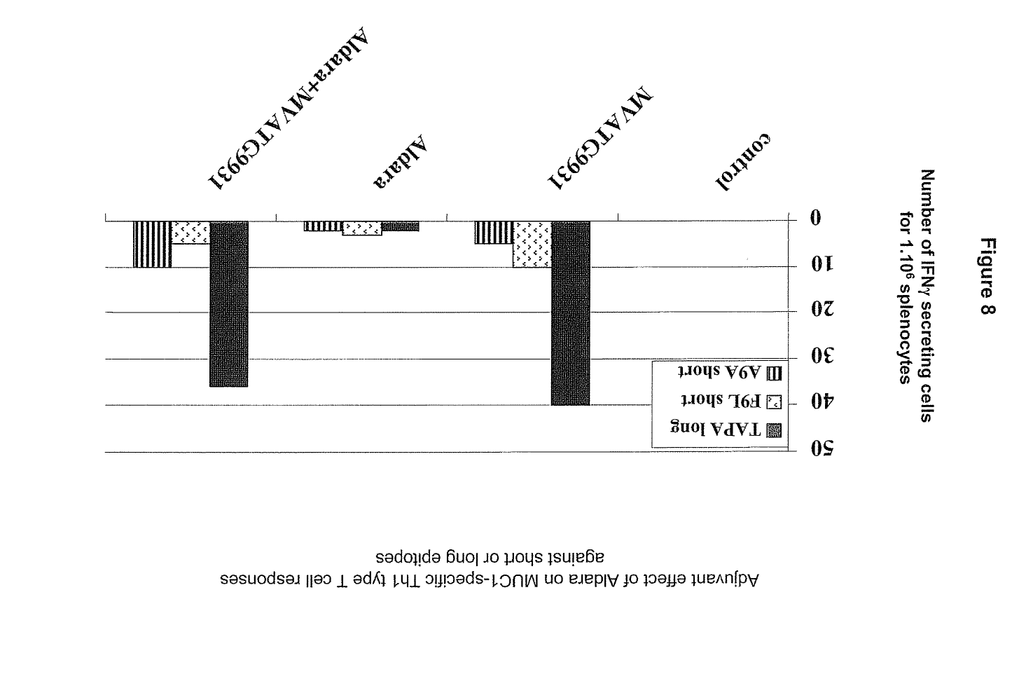

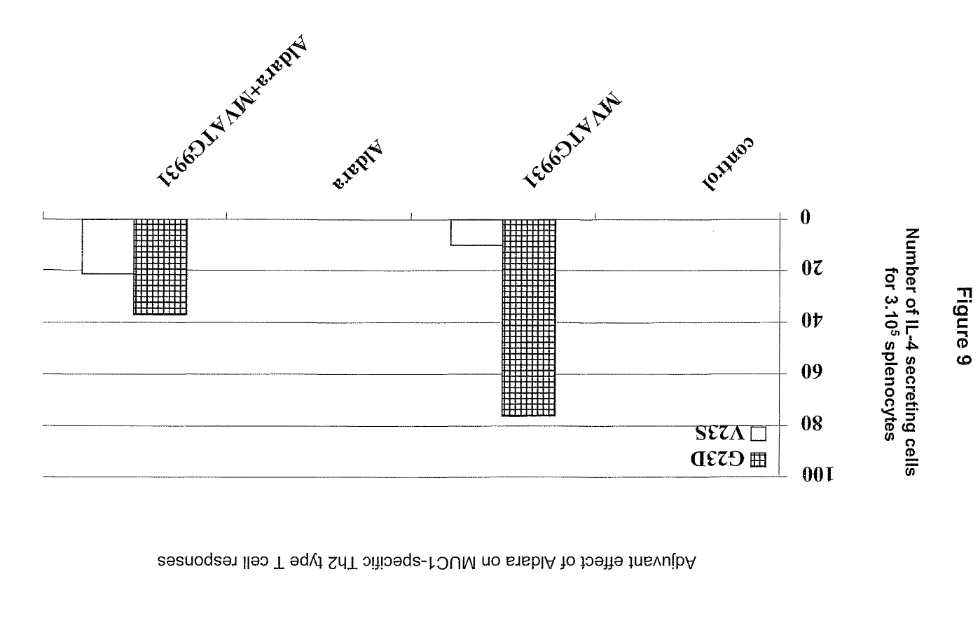

In another set of experiments, the number of MUC1-specific IL-4 secreting cells was enumerated using an ELISPOT assay. MUC1 restricted peptides were used to monitored both CD4 and CD8 T cell response after immunization. We have observed that pre-treatment with a topical administration of Aldara reduce significantly the number of Th2-based T cell response obtained with MVATG9931 alone (FIG. 9).

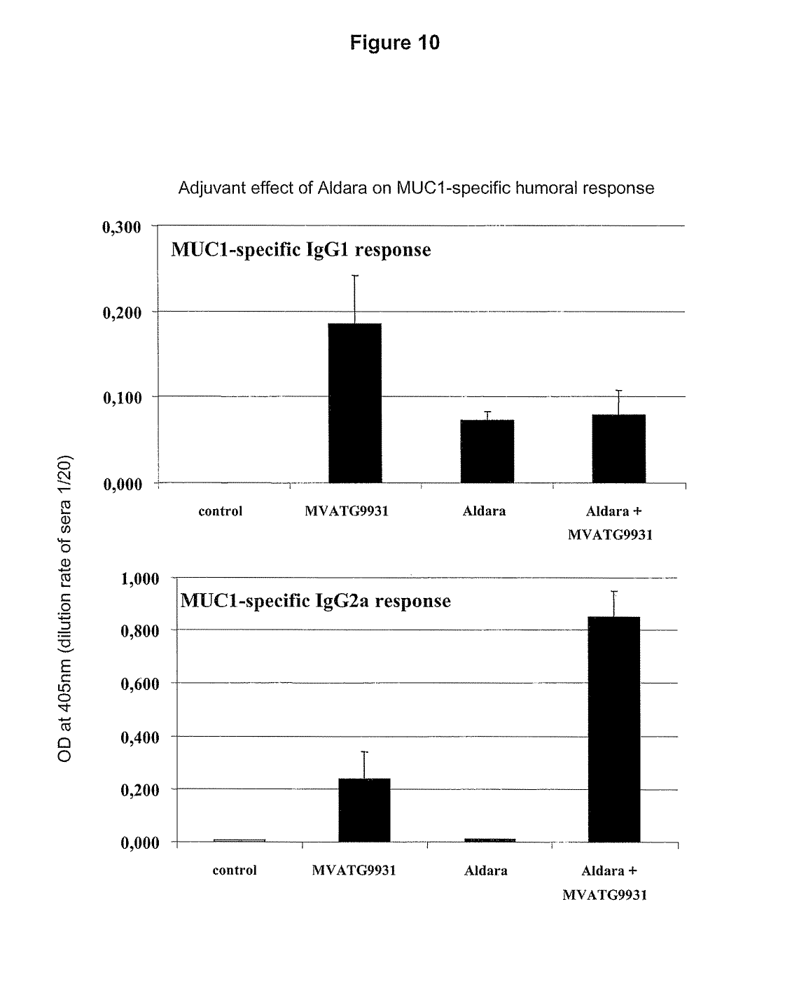

Finally the measure of the humoral response against the MUC1 antigen was also performed by ELISA. In order to better characterize the type of response induced by the combination Aldara™+MVATG9931, the IgG isotype switch was analysed. MUC1-specific IgG1 and IgG2a were detected (FIG. 10). We have observed that pre-treatment by a topical administration of Aldara™ induce a typical Th1 type response (higher IgG2a titre than IgG1) which could be implicated in the efficacy of the treatment.

C—Conclusions and Discussions

These experiments demonstrate for the first time that topical application of Aldara™ can improve the therapeutic efficacy (improve the immune response) of a MVA-based vaccine towards antigens.

1-4. (canceled)

5. A recombinant viral vaccine comprising:
(i) at least one recombinant poxviral vector expressing in vivo at least one heterologous nucleotide sequence; and
(ii) at least one 1H-imidazo[4,5-c]quinolin-4-amine derivative.

6. The recombinant viral vaccine of claim 5, wherein said at least one heterologous nucleotide sequence encodes an antigen.

7. A kit for vaccination, comprising:
(i) a container comprising at least one recombinant poxviral vector expressing in vivo at least one heterologous nucleotide sequence; and
(ii) a container comprising at least one 1H-imidazo[4,5-c]quinolin4-amine derivative.

8. The kit of claim 7, wherein said at least one heterologous nucleotide sequence encodes an antigen.

9. The recombinant viral vaccine of claim 5, wherein said poxviral vector is a highly attenuated vaccinia virus strain.

10. The recombinant viral vaccine of claim 9, wherein said highly attenuated vaccina virus strain is a highly attenuated vaccinia virus strain obtained from the Copenhagen or the Wyeth strain.

11. The recombinant viral vaccine of claim 5, wherein said 1H-imidazo[4,5-c]quinolin-4-amine derivative is a compound defined by one of the following general formulae I-V: 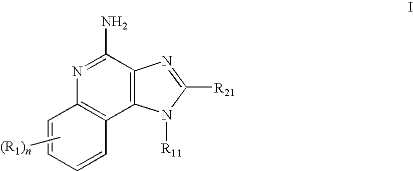
or analogues, solvates or salts thereof,
wherein
Rᵢᵢ is selected from the group consisting of straight or branched alkyl, hydroxyalkyl, acyloxyalkyl, benzyl, (phenyl)ethyl and phenyl, said benzyl, (phenyl)ethyl or phenyl substituent being optionally substituted on the benzene ring by one or two moieties selected, independently from one another, from the group consisting of C₁₋₄ alkyl moiety, C₁₋₄ alkoxy moiety and halogen, with the proviso that if said benzene ring is substituted by two of said moieties, then said moieties together contain no more than 6 carbon atoms;
R₂₁ is selected from the group consisting of hydrogen, C₁₋₈ alkyl moiety, benzyl, (phenyl)ethyl and phenyl, the benzyl, (phenyl)ethyl or phenyl substituent being optionally substituted on the benzene ring by one or two moieties selected, independently from one another, from the group consisting of C₁₋₄ alkyl moiety, C₁₋₄ alkoxy moiety and halogen, with the proviso that when the benzene ring is substituted by two of said moieties, then the moieties together contain no more than 6 carbon atoms;
and each Rᵢ is selected, independently from one another, from the group consisting of hydrogen, C₁₋₄ alkoxy moiety, halogen and C₁₋₄ alkyl moiety, and n is an integer from 0 to 2, with the proviso that if n is 2, then said R₁ groups together contain no more than 6 carbon atoms; 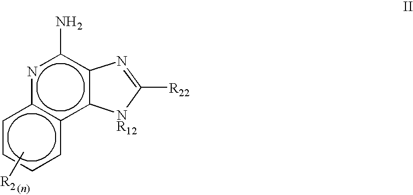
or analogues, solvates or salts thereof,
wherein
R₁₂ is selected from the group consisting of straight or branched C₂₋₁₀ alkenyl and substituted straight or branched C₂₋₁₀ alkenyl, wherein the substituent is selected from the group consisting of straight or branched C₁₋₄ alkyl moiety and C₃₋₆ cycloalkyl moiety; and C₃₋₆ cycloalkyl moiety substituted by straight or branched C₁₋₄ alkyl moiety; and
R₂₂ is selected from the group consisting of hydrogen, straight or branched C₁₋₈ alkyl moiety, benzyl, (phenyl)ethyl and phenyl, the benzyl, (phenyl)ethyl or phenyl substituent being optionally substituted on the benzene ring by one or two moieties selected, independently from one another, from the group consisting of straight or branched C₁₋₄ alkyl moiety, straight or branched C₁₋₄ alkoxy moiety, and halogen, with the proviso that when the benzene ring is substituted by two such moieties, then the moieties together contain no more than 6 carbon atoms;
and each R₂ is selected, independently from one another, from the group consisting of straight or branched C₁₋₄ alkoxy moiety, halogen, and straight or branched C₁₋₄ alkyl moiety, and n is an integer from zero to 2, with the proviso that if n is 2, then said R₂ groups together contain no more than 6 carbon atoms; 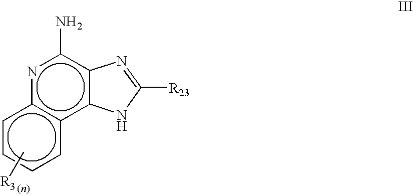
or analogues, solvates or salts thereof,
wherein
R₂₃ is selected from the group consisting of hydrogen, straight or branched C₁₋₈ alkyl moiety, benzyl, (phenyl)ethyl and phenyl, the benzyl, (phenyl)ethyl or phenyl substituent being optionally substituted on the benzene ring by one or two moieties selected, independently from one another, from the group consisting of straight or branched C₁₋₄ alkyl moiety, straight or branched C₁₋₄ alkoxy moiety, and halogen, with the proviso that when the benzene ring is substituted by two such moieties, then the moieties together contain no more than 6 carbon atoms;
and each R₃ is selected, independently from one another, from the group consisting of straight or branched C₁₋₄ alkoxy moiety, halogen, and straight or branched C₁₋₄ alkyl moiety, and n is an integer from zero to 2, with the proviso that if n is 2, then said R₃ groups together contain no more than 6 carbon atoms; 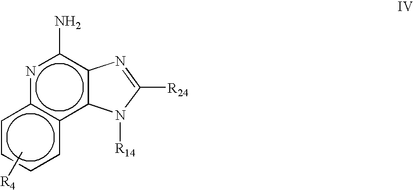
or analogues, solvates or salts thereof,
wherein
R₁₄ is —CHR34R₄₄ wherein R₄₄ is hydrogen or a carbon-carbon bond, with the proviso that when R₄₄ is hydrogen R₃₄ is C₁₋₄ alkoxy moiety, C₁₋₄ hydroxyalkoxy moiety, C₂₋₁₀ 1-alkynyl moiety, tetrahydropyranyl, alkoxyalkyl wherein the alkoxy moiety contains one to four carbon atoms and the alkyl moiety contains one to four carbon atoms, 2-, 3-, or 4-pyridyl, and with the further proviso that when R₄₄ is a carbon-carbon bond R₄₄ and R₃₄ together form a tetrahydrofuranyl group optionally substituted with one or more substituents selected, independently from one another, from the group consisting of hydroxy and C₁₋₄ hydroxyalkyl moities;
R₂₄ is selected from the group consisting of hydrogen, C₁₋₄ alkyl, phenyl, wherein the phenyl is optionally substituted by one or two moieties selected, independently from one another, from the group consisting of straight or branched C₁₋₄ alkyl moiety, straight or branched C₁₋₄ alkoxy moiety, and halogen;
and R₄ is selected from the group consisting of hydrogen, straight or branched C₁₋₄ alkoxy moiety, halogen, and straight or branched C₁₋₄ alkyl moiety; 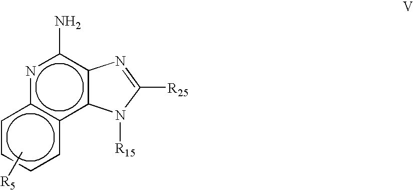
or analogues, solvates or salts thereof,
wherein
R₁₅ is selected from the group consisting of hydrogen; straight or branched C₁₋₁₀ alkyl moiety and substituted straight or branched C₁₋₁₀ alkyl moiety, wherein the substituent is selected from the group consisting of C₃₋₆ cycloalkyl and C₃₋₆ cycloalkyl substituted by straight or branched C₁₋₄ alkyl moiety; straight or branched C₂₋₁₀ alkenyl and substituted straight or branched C₂₋₁₀ alkenyl moiety, wherein the substituent is selected from the group consisting of C₃₋₆ cycloalkyl and C₃₋₆ cycloalkyl substituted by straight or branched C₁₋₄ alkyl moiety; C₁₋₆ hydroxyalkyl; alkoxyalkyl wherein the alkoxy moiety contains one to about four carbon atoms and the alkyl moiety contains one to about six carbon atoms; acyloxyalkyl wherein the acyloxy moiety is alkanoyloxy of two to about four carbon atoms or benzoyloxy, and the alkyl moiety contains one to about six carbon atoms; benzyl; (phenyl)ethyl; and phenyl; said benzyl, (phenyl)ethyl or phenyl substituent being optionally substituted on the benzene ring by one or two moieties selected, independently from one another, from the group consisting of C₁₋₄ alkyl moiety, C₁₋₄ alkoxy moiety, and halogen, with the proviso that when said benzene ring is substituted by two of said moieties, then the moieties together contain no more than six carbon atoms;
R₂₅ is 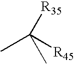
wherein
R₃₅ is selected from the group consisting of C₁₋₄ alkoxy moiety, alkoxyalkyl wherein the alkoxy moiety contains one to about four carbon atoms and the alkyl moiety contains one to about four carbon atoms; C₁₋₄ haloalkyl moiety; alkylamido wherein the alkyl group contains one to about four carbon atoms; amino; amino substituted with C₁₋₄ alkyl or C₁₋₄ hydroxyalkyl; azido; C₁₋₄ alkylthio;
R₅₅ and R₄₅ are selected, independently from one another, from the group consisting of hydrogen, C₁₋₄ alkyl moiety, phenyl, wherein said phenyl is optionally substituted by one or two moieties selected, independently from one another, from the group consisting of straight or branched C₁₋₄ alkyl moiety, straight or branched C₁₋₄ alkoxy moiety, and halogen; and
R₅ is selected from the group consisting of hydrogen, straight or branched C₁₋₄ alkoxy moiety, halogen, and straight or branched C₁₋₄ alkyl containing moiety.

12. The recombinant viral vaccine of claim 5, wherein said 1H-imidazo[4,5-c]quinolin-4-amine derivative is a compound defined by the following general formula VI: 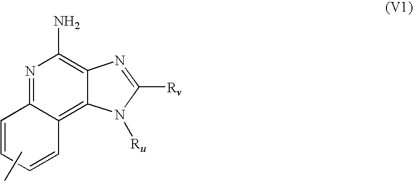
or analogues, solvates or salts thereof,
wherein
Rₜ is selected from the group consisting of hydrogen, straight or branched C₁₋₄ alkoxy moiety, halogen, and straight or branched C₁₋₄ alkyl;
Rᵤ is 2-methylpropyl or 2-hydroxy-2-methylpropyl; and
Rᵥ is hydrogen, C₁₋₆ alkyl, or alkoxyalkyl wherein the alkoxy moiety contains one to about four carbon atoms and the alkyl moiety contains one to about four carbon atoms.

13. The recombinant viral vaccine of claim 5, wherein said heterologous nucleotide sequence encodes one or more of all or part of the HPV-E1, E2, E3, E4, E5, E6, E7, E8, L1, and L2 antigens.

14. A method of enhancing an immune response to an antigen in a patient, said method comprising administering to said patient:
(i) at least one recombinant viral vector expressing in vivo at least one heterologous nucleotide sequence encoding an antigen; and
(ii) at least one 1H-imidazo[4,5-c]quinolin-4-amine derivative;
thereby enhancing an immune response to said antigen.

15. The method of claim 14, wherein said 1H-imidazo[4,5-c]quinolin-4-amine derivative is administered independently of and prior to administration of said recombinant viral vector.

16. The method of claim 14, wherein said 1H-imidazo[4,5-c]quinolin-4-amine derivative and said recombinant viral vector are administered simultaneously.

17. The method of claim 14, wherein said recombinant viral vector is a recombinant poxviral vector.

18. The method of claim 14, wherein said heterologous nucleotide sequence encodes one or more of all or part of the HPV-E1 E2, E3, E4, E5, E6, E7, E8, LI, and L2 antigens.
